(19) United States
(12) Patent Application Publication
(10) Pub. No.: US 2002/0032187 A1
(43) Pub. Date: Mar. 14, 2002

(54) Method for stimulating bone formation

(75) Inventor: Fred H. Drake, Glenmoore, PA (US)

Correspondence Address: SMITHKLINE BEECHAM CORPORATION, Corporate Intellectual Property - UW2220, P.O. Box 1539, King of Prussia, PA, 19406-0939, US

(73) Assignee: SmithKline Beecham Corporation

(21) Appl. No.: 09/956,659

(22) Filed: Sep. 20, 2001

Related U.S. Application Data
(63) Continuation of application No. 09/639,347, filed on Aug. 16, 2000.
Application No. 09/639,347 is a continuation of application No. 09/284,055, filed on Apr. 7, 1999, now abandoned.
Application No. 09/284,055 is a 371 of international application No. PCT/US97/18178, filed on Oct. 7, 1997.
(60) Provisional application No. 60/027,764, filed on Oct. 7, 1996.

(51) Int. Cl. A61K 31/553; A61K 31/554; A61K 31/55; A61K 31/235

(52) U.S. Cl. 514/211.06; 514/221; 514/212.07; 514/532

(57) ABSTRACT

A method for stimulating bone formation by administering integrin binding compounds which cause the release of osteocalcin from osteoblasts is disclosed.

### FIELD OF THE INVENTION

This invention relates to a new method for treating bone-related disorders with compounds which stimulate bone-formation.

### BACKGROUND

The bone matrix consists of inorganic mineral and organic matrix. The mineral, which constitutes 65% of bone, is mainly calcium and phosphorous in hydroxyapatite crystals. The matrix, which constitutes 35% of the bone tissue, consists primarily of type I collagen (90%) and an assortment of non-collagenous proteins including osteopontin, bone sialoprotein, fibronectin, thrombospondin, osteocalcin, osteonectin, proteoglycans, and glycosaminoglycans. Osteocalcin appears to be restricted to the cells of calcified tissues and is regulated by 1,25(OH)₂ D3. It has been suggested that serum osteocalcin may be a marker for bone formation. See Hauschka et al., Phys. Rev., 69, 990 (1989). and Charles et al., Calcified Tissue International, 51,406 (1992).

Bone is extremely dynamic. At the tissue level it is continuously being resorbed and rebuilt in order to maintain the strength and vitality of skeletal tissue and to meet the demands of mineral homeostasis. Resorption and formation are carried out by osteoclasts and osteoblasts, respectively, at discrete foci in the skeleton. These foci, or remodeling units, represent sites for a cycle of resorption followed by new bone formation. The remodeling cycle begins when an osteoclast initiates the resorptive phase. Over a period of weeks these cells resorb bone until a signal, as yet undefined, stops the resorptive process and initiates the formation phase by recruitment of osteoblasts to the site. During growth and early adulthood, bone modeling and remodeling result in a positive balance (i.e., net bone formation). However, later in life there is an apparent uncoupling of the resorption and formation events resulting in a negative bone balance (i.e., net bone loss). A number of diseases of the skeleton are characterized by such an imbalance, including osteoporosis, hyperparathyroidism, hypercalcemia of malignancy, and Paget's disease.

Bone formation occurs by an ordered process of osteoblast-mediated matrix deposition and mineralization. These events are associated with commitment of osteoblast precursors to a differentiation pathway that leads through a series of stages to mature osteoblasts; these stages are defined by changes in expression of markers of differentiation such as alkaline phosphatase, type I collagen, and osteocalcin.

Most current approaches to restoring the balance between formation and resorption are aimed at inhibiting osteoclastic bone resorption. If osteoporosis is detected early, antiresorptive therapy is effective at slowing the progression of the disease. However, when a substantial amount of bone is already lost, inhibition of bone resorption alone may not be sufficient to remove the risk of fractures. Accordingly, therapies which enhance bone formation are needed to regenerate the bone and decrease the susceptibility to fracture.

Integrins are a family of cell surface receptors that are involved in both cell-matrix and cell-cell interactions. The receptors are heterodimers composed of one α and one β subunit, each of which contain large extracellular domains, a single transmembrane domain, and relatively short cytoplasmic tail. At least 14 α and 8 β subunits have been identified, although not all α and β subunits appear to interact to form functional receptors. At least 20 combinations have been described. Hynes, R. O., Cell, 69, 11 (1992).

Studies have indicated that the attachment of osteoclasts to the bone matrix is mediated through integrin receptor. For instance, Davies, et al., J. Cell Biol., 1989, 109, 1817, disclose that the osteoclast functional antigen, which is implicated in the regulation of bone resorption, is biochemically related to the vitronectin receptor. The vitronectin receptor, or the αᵥβ₃ integrin, is known to bind to bone matrix proteins, such as osteopontin, bone sialoprotein and thrombospondin, which contain the tri-peptide Arg-Gly-Asp (or RGD) motif. Thus, Horton, et al., Exp. Cell Res. 1991, 195, 368, disclose that RGD-containing peptides and an anti-vitronectin receptor antibody (23C6) inhibit dentine resorption and cell spreading by osteoclasts. In addition, Sato, et al., J. Cell Biol. 1990, 111, 1713 disclose that echistatin, a snake venom peptide which contains the RGD sequence, inhibits attachment of osteoclasts to bone. Bertolini et al., J. Bone Min. Res., 6, Sup. l, S146, 252 have shown that cylco-S,S-N^{α}-actyl-cysteinl-N^{α}-methyl-argininyl-glycyl-aspartyl-penicillarnine inhibits osteoclast attachment to bone. EP 528 587 and 528 586 report substituted phenyl derivatives which inhibit osteoclast mediated bone resorption.

There is limited information on the role of integrins in osteoblast development or function. Several groups have used immunolocalization and in situ hybridization in an attempt to characterize the integrins present on these cells, and there appear to be clear differences between the integrin expression in osteoblasts and osteoclasts. See Clover et al., J. Cell Sci., 103, 267 (1992). There is also the suggestion of heterogeneity of expression within the osteoblasts.

Accepted treatments which stimulate bone formation over prolonged periods have not been identified. Sodium fluoride, parathyroid hormone and strontium salts have been reported to stimulate bone formation in vivo, while zeolite A and various growth factors, such as IGF-1, IGF-2, TGF-β and bone morphogenic proteins, have been reported to stimulate osteoblast proliferation and differentiation in vitro. Nevertheless, there remains a need for new and effective methods to induce bone formation.

### DETAILED DESCRIPTION

It has now been discovered that bone formation may be stimulated by treating osteoblasts with compounds which bind to a receptor on osteoblasts. The ability of compounds to bind this receptor and to induce bone formation may be demonstrated by the release of osteocalcin from cell cultures of osteoblasts, in vitro mineralization of bone tissue, enhanced matrix deposition, and in vivo bone formation.

Although osteocalcin has been associated with osteoblast activity, it has now been discovered that an osteocalcin release assay may be used to identify compounds which stimulate mineralization and bone formation in vivo. Thus, in one respect, this invention is an assay for identifying compounds which stimulate bone formation or mineralization which comprises treating osteoblasts with a compound and assaying for an increase in osteocalcin release. This discovery has led to the further discovery that small molecules may act directly upon osteoblasts to induce bone formation.

The osteoblasts may be derived from a natural source, such as by isolating and culturing osteoblasts from the bone tissue of a mammal, or from an immortalized cell line, such as an osteosarcoma, or by differentiation of precursor cells, such as stromal cells, into osteoblasts. Rat, mouse and human cells are particularly useful for this, although other mammals would also be useful. In a particularly useful embodiment of this invention ROS 17/2.8 osteoblasts are useful.

Since osteoblasts generally produce osteocalcin at a basal level, one needs to quantitate the increase in osteocalcin production to obtain an indication of the extent of osteoblast activation. Generally, the compounds of this invention, like endogenous mediators of bone production (such as parathyroid hormone and di-(OH)2-vitarnin D3), will induce a maximal two to three fold increase in osteocalcin production in a dose-related fashion. Thus, one may construct a dose-response curve and determine from the curve an EC50 for osteocalcin release. Accordingly, as used herein, “assaying” for osteocalcin release mean measuring the increase in osteocalcin production relative to the basal and maximal level of osteocalcin release caused by a compound of this invention. A convenient measure of the activity of a compound to stimulate osteoblasts and bone formation is the EC50 of a compound for eliciting osteocalcin release.

A convenient method for quantitating osteocalcin release is radioimmunoassay. Antibodies to osteocalcin and kits for quantitating osteocalcin are commercially available, or may be prepared by routine methods.

In another aspect, this invention is a method for stimulating bone formation which comprises administering a compound which causes an increase in osteocalcin release. Alternatively, this is a method for stimulating bone formation which comprises administering an integrin-binding compound. Preferably, the compound will cause at least a two fold increase in osteocalcin production, and will have an EC50 of less than 10 uM, more preferably below I uM and most preferably below 0.1 uM. Increased bone production is a clear benefit in disease states wherein there is a deficiency of mineralized bone mass or remodeling of bone is desired, such as osteoporosis, fracture healing, and the prevention of bone fractures. Diseases and metabolic disorders which result in loss of bone structure would also benefit from such treatment. For instance, hyperparathyroidism, Paget's disease, hypercalcemia of malignancy, osteolytic lesions produced by bone metastasis, and bone loss due to immobilization or sex hormone deficiency, could benefit from administering a compound of this invention.

“Compounds” as used herein is intended to mean any chemical compound, other than a natural endogenous ligand, such as vitamin D3 or parathyroid hormone, which stimulates osteocalcin release. Thus, the compound may be a peptide, protein, antibody or a small organic molecule. In a particular embodiment, the compound binds to an integrin receptor. The interaction that mediates the osteocalcin release and bone formation is believed to be an interaction with an integrin or a group of integrin receptors, since compounds which contain the -Arg-Gly-Asp- sequence, such as cyclo(Ara-Gly-Asp-D-Phe-Vall and Gly-Arg-Gly-Asp-Ser, are able to stimulate osteoblasts and cause bone formation. The -Arg-Gly-Asp- motif is commonly found in integrin receptor ligands. In addition, other compounds that are known to bind to integrin receptors have also been found to be useful in this invention. Accordingly, in a preferred embodiment compounds which bind to integrin receptors are of particular interest for use in the method of this invention. Integrin-binding compounds that useful in this invention are conveniently identified by assaying their ability to induce osteocalcin release from osteoblasts. Preferably the compounds will stimulate osteocalcin release with an EC50 of less than 10 uM, more preferably at less than 1 uM. Most preferably the compounds will have an EC50 of less than 0.1 uM.

Certain compounds which bind to integrins, such as the αᵥβ₃ (vitronectin) receptor, have been found to be especially useful for stimulating osteocalcin release, and this class of compounds are particularly attractive ligands for testing in the assay of this invention. Although there is no strict correlation between αᵥβ₃ binding and the ability to induce the desired bone-forming activity, the osteocalcin release assay may be used to distinguish those compounds which do promote bone formation. Thus, in a preferred embodiment, this invention is a method for stimulating bone formation which comprises administering an integrin binding compound, particularly an αᵥβ₃ binding compound, which causes the release of osteocalcin. Compounds which have a Ki for binding the αᵥβ₃ receptor of less than 2 uM, more often less than I uM are often active in the assay. Vitronectin receptor antagonists are often also potent stimulators of osteocalcin release. It should be appreciated, however, that the effect is not necessarily due to interaction with the vitronectin receptor, since pre-treatment of osteoblasts with neutralizing antibodies specific for the vitronectin receptor does not effect the release of osteocalcin, and does not mimic or block bone formation induced by the active compounds of this invention. Typically, compounds which have the desired bone-forming activity will bind to the α_{IIb}β₃ receptor with a Ki of greater than 10 uM. Compounds which have been found to elicit osteocalcin release have also been shown to induce mineralization in osteoblastic cell lines from murine, rat and human origin. Thus, Table 1 illustrates the generality of the response to various osteoblastic cell lines to the osteocalcin-releasing compounds cyclo(Arg-Gly-Asp-D-Phe-Val) and 7-[(((2-benzimidazolyl)methyl)amino)carbonyl]-2,3,4,5-tetrahydro4-methyl-3-oxo-1H-1,4-benzodiazepine-2-acetic acid:

TABLE 1

| Cell Type | Source | Species | Response |
|---|---|---|---|
| MC3T3 E1 | osteosarcoma | murine | + |
| PROB's | new-born calvaria | rat | + |
| SaOS-1 | osteosarcoma | human | + |
| HOB's | trabecular bone | human | + |
| TF-274 | bone marrow | human | + |
| | (stromal-derived) | | |
| L929 | fibroblast (control) | murine | − |

and also confirms that cells of non-osteoblastic origin (e.g., L929 cells) do not mineralize under similar conditions.

Compounds which bind to integrin receptors are a well known class of compounds. Certain of such compounds are disclosed in the art, for instance, in Blackburn, et al., WO 93/08174; Bondinell, er al., WO 95/18619; Bondinell, et al., WO 94/14776; Blackburn. et al. WO 95/04057; Ali et al., WO 96/00730, Keenan et al., WO 96/00574; Bondinell er al., WO 97/01540; Miller er al., WO 97/24119, All et al., WO 97/24122; and All et al., WO 97/24124, and in references cited therein.

A particularly useful class of compounds are those given by the general formula (I):

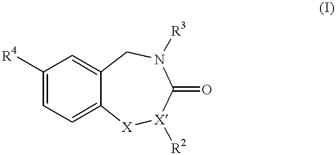

wherein

X-X′ is NR¹—CH, NC(O)R¹—CH, N═C, CR¹═C, CHR¹—CH, O—CH or S—CH;

R¹ is H, C₁₋₆alkyl or Ar—C₁₋₆alkyl;

R² is (CH₂)ₙCO₂R′;

R³ is H, C₁₋₆alkyl, Ar—C₀₋₆alkyl, Het—C₀₋₆alkyl, or C₃₋₆cycloalkyl-C₀₋₆alkyl;

R⁴ is W-(Q′)ₚ-(CR′₂)_{q}-U-(CR′₂)ₛ-;

R⁵ and R⁶ are H, C₁₋₆alkyl, Ar—C₀₋₆alkyl, Het—C₀₋₆alkyl or C₃₋₆cycloalkyl-C₀₋₆alkyl;

R′ is H, C₁₋₆alkyl, C₃₋₇cycloalkyl-C₀₋₄alkyl or Ar-C₀₋₄alkyl;

Q′ is NR⁵, S or CR⁵;

U is NR⁶C(O), C(O)NR⁶, CH₂CO, COCH₂, CH═CH, C≡C, CH₂—CH₂, O—CH₂, CH₂—O, O, S, NR⁶, C(O), CH₂ or CH₂OCONR¹;

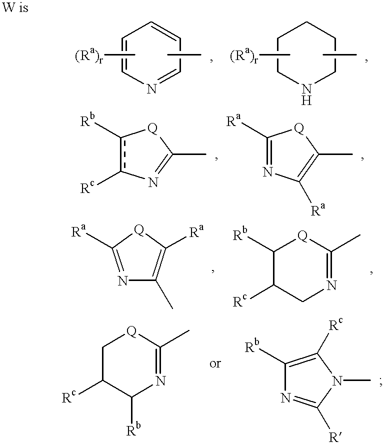

Q is NR′, O or S;

[symbol] indicates a single or double bond:

R^{a} is independently H, C₁₋₆alkyl, Ar-C₀₋₆alkyl, Het-C₀₋₆alkyl, or C₃₋₆cycloalkyl-C₀₋₆alkyl, halogen, OR¹, SR¹, COR¹, OH, NO₂, N(R¹)₂, CO(NR¹)₂, CH₂N(R¹)₂ or R¹HN—C(=NH);

R^{b} and R^{c} are independently selected from H, C₁₋₆alkyl, Ar-C₀₋₆alkyl, Het-C₀₋₆alkyl, or C₃₋₆cycloalkyl-C₀₋₆alkyl, halogen, OR¹, SR¹, COR¹, OH, NO₂, N(R¹)₂, CO(NR¹)₂, CH₂N(R¹)₂, or R^{b} and R^{c} are joined together to form a five or six membered aromatic or non-aromatic ring, optionally substituted by halogen, C₁₋₄alkyl, OR¹, SR¹, COR¹, OH, NO₂, N(R¹)₂, CO(NR¹)₂, CH₂N(R¹)₂ or R¹HN—C(=NH); or

n is 1 or 2;

p is 0 or 1;

q is 0, 1, 2 or 3;

r is 0, 1 or 2;

s is 0, or 2; or a pharmaceutically acceptable salt thereof.

Another group of compounds which have been found to be useful are of formula (II):

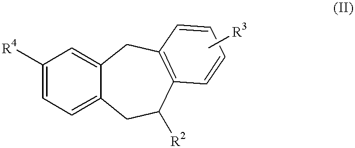

wherein R², R³ and R⁴ are as defined for formula (I).

Certain peptides containing the Arg-Gly-Asp- sequence, or modifications of this sequence, have also been founds to stimulate the release of osteocalcin. Representative peptides active in this invention are: cyclo[Arg-Gly-Asp-D-Phe-Val]; Benzyl-(N-methyl)Arg-Gly-Asp-NHPh; Benzyl-(N-methyl)Arg-Gly-Asp-NH-(2 or 4 chloro)Ph; Gly-Arc-Gly-Asp-Ser-OH; and the snake venom peptide eichistatin.

In addition, other integrin receptor ligands such as:

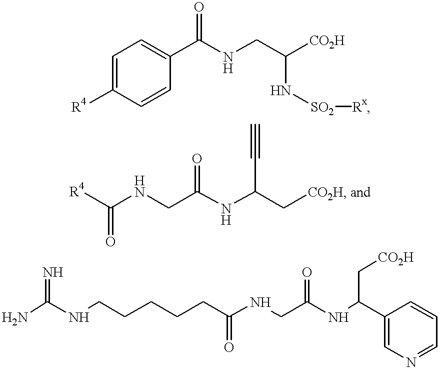

wherein R⁴ is as above for formula (I), and R^{x} is Ar or C₁₋₆alkyl, particularly phenyl or n-butyl, also cause the release of osteocalcin. These compounds demonstrate the general applicability of integrin binding ligands for use in the method and for testing in the assay.

Suitably in formula (1), X-X′ is CH₂—CH or NR¹CH.

Suitably, R² is CH₂CO₂R′; preferably CO₂H.

Suitably, R³ is H, C₁₋₆alkyl, CF₃CH₂ or C₁₋₄alkoxyC₁₋₄alkyl, or benzyl or phenylethyl optionally substituted by one to three CF₃, CF₃O, NO₂, CN, methoxy, halo, —O—CH₂—O—, C₁₋₄alkyl.

R⁴ is W-(Q′)ₚ-(CR′₂)_{q}—U—.

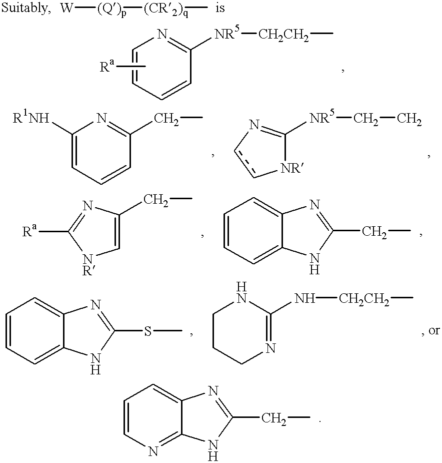

Suitably, R⁵ is H′ or methyl.

Suitably, R⁶ is H or C₁₋₆alkyl optionally substituted by amino, CN, pyridyl, benzimidazolyl, Ar—CONH—, biotin-C₁₋₆alkyl-CONH. Preferably, R⁶ is H or methyl.

Suitably, R^{a} is H, methyl, methoxy or NHR¹.

Specific compounds of this invention are listed in the examples.

Representative compounds of formula (I) and (II) are:

(+/−)-7-[[(2-Methyl benzimidazoloyl)amino]carbonyl]-2,3,4,5-tetrahydro-4-methyl-3-oxo-1H-1,4-benzodiazepine-2-acetic acid;

(+/−)-7-[[(2-Methyl benzimidazoloyl)amino]carbonyl]-2,3,4,5-tetrahydro4methyl-3-oxo-1H-1,4-benzodiazepine-2-acetic acid;

(S)-(-)-7-[[[(2-Benzimidazolyl)methyl]amino]carbonyl]-2,3,4,5-tetrahydro-4-methyl-3-oxo-1H-1,4-benzodiazepine-2-acetic acid;

(+/−)-7-[[[(2-Methylbenzimidazolyl](N-methyl)amino]carbonyl]-2,3,4,5-tetrahydro-4-methyl-3-oxo-1H-1,4-benzodiazepine-2-acetic acid trifluoroacetate salt;

(+/−)-7-[[(Benzimidazol-2-yl)methyl]amino]carbonyl]-3-oxo-4-(2-phenylethyl)-2,3,4,5-tetrahydro-1H-1,4-benzodiazepine-2-acetic acid;

(+/−)-7-[[[(Benzimidazol-2-yl)methyl]amino]carbonyl]-3-oxo-4-isopropyl-2,3,4,5-tetrahydro-1H-1,4-benzodiazepine-2-acetic acid trifluoroacetate salt;

(+/−)-2-Methyl-3-oxo-8-([(2-benzimidazoyl)methylamino]carbonyl)-2,3,4,5-tetrahydro-2-benzazepine-4-acetic acid;

(+/−)-8-[[(2-Benzimidazoylmethyl)(N-methyl)amino]carbonyl]-2,3,4,5-tetrahydro-2-methyl-3-oxo-2-benzapine-4-acetic acid hydrochloride salt;

(+/−)-7-[[(2-Benzimidazolylmethyl](N-methyl)amino]carbonyl]-2,3,4,5-tetrahydro-3-oxo-4-(2-phenylethyl)-1H-1,4-benzodiazepine-2-acetic acid;

(S)-7-[[(1H-Benzimidazol-2-ylmethyl)methylamino]carbonyl]-2,3,4,5-tetrahydro-4-methyl-3-oxo-1H-1,4-benzodiazepine-2-acetic acid=(S)-(-)-7-[[N-alpha-[(2-Methylbenzimidazolyl)(N-methyl)amino]carbonyl]-2,3,4,5-tetrahydro-4-methyl-3-oxo-1H-1,4-benzodiazepine-2-acetic acid;

(+/−)-7-[[[( 1-N-methyl)-2-methylbenzimidazolyl](N-methyl)amino]carbonyl]-2,3,4,5-tetrahydro-4-methyl-3-oxo-1H-1,4-benzodiazepine-2-acetic acid;

(S)-7-[[(2-Benzimidazol-2-ylmethyl](N-methyl)amino]carbonyl]-2,3,4,5-tetrahydro-3-oxo-4-(2-phenylethyl)-1H-1,4-benzodiazepine-2-acetic acid;

(-)-7-[[[5,6-MethylenedioxybenzimidazoI-2-ylmethyl]aminomethyl]carbonyl]-2,3,4,5-tetrahydro-4-methyl-3-oxo-1,4-benzodiazepine-2-acetic acid;

(2S)-7-[[[N-Butyl-N-(benzimidazol-2-ylmethyl]amino]carbonyl]-3-oxo-4-methyl-2,3,4,5-tetrahydro-1H-1,4-benzodiazepine-2-acetic acid;

(-)-7-[[[Imidazo[4,5B]pyridyl-2-ylmethyl]aminomethyl]carbonyl]2,3,4,5-tetrahydro-4-methyl-3-oxo-1,4-benzodiazepine-2-acetic acid;

(2S)-[[[N-Phenylethyl-N-(benzimidazol-2-yl)methyl amino]carbonyl]-3-oxo-4-methyl-2,3,4,5-tetrahydro-1H-1,4-benzodiazepine-2-acetic acid;

(+/−)-7-[[[(2-Methyl)benzimidazolyl](N-methyl)amino]carbonyl]-2,3,4,5-tetrahydro-3-oxo-1H-1,4-benzodiazepine-2-acetic acid trifluoroacetate;

(+/−)-7-[[[N-Benzimidazol-2-yl]methyl]amino]methyl]-4-methyl-3-oxo-2,3,4,5-tetrahydro-1H-1,4-benzodiazepine-2-acetic acid bis(trifluoroacetate);

(+/−)-7-[[[Imidazo[4,5-B]pyridyl-2-ylmethyl]aminomethyl]carbonyl]2,3,4,5-tetrahydro-4-isoproyl-3-oxo-1,4-benzodiazepine-2-acetic acid;

(-)-7-[[[Imidazo(4,5-B ]-6-methylpyridyl-2-ylmethyl aminomethyl]carbonyl]-2,3,4,5-tetrahydro-4-methyl-3-oxo-benzodiazepine-2-acetic acid.;

(+/−)-2,3,4,5-Tetrahydro-7-[[[(benzimidazol-2-yl)methyl]methyl]amino]carbonyl]-4-(2-methoxyethyl)-3-oxo-1H-1,4-benzodiazepine-2-acetic acid;

(+/−)-2,3,4,5-Tetrahydro-7-[[[imidazo[4,5-B]pyridyl-2-yl]methyl]methylamino]carbonyl]-4-(2-methoxyethyl)-3-oxo-1H-1,4-benzodiazepine-2-acetic acid trifluoroacetate salt;

(+/−)-7-[[(2-Benzimidazol-2-ylmethyl)amino]carbonyl]-2,3,4,5-tetrahydro-3-oxo-4-[2-(3′,4′-methylenedioxyphenyl)ethyl]-1H-1,4-benzodiazepine-2-acetic acid; and

(±)-2,3,4,5-Tetrahydro-3-oxo-4-(phenylethyl)-7-[[[2-[2-(pyridinyl)amino]ethyl]amino]carbonyl]-1H-1,4-benzodiazepine-2-acetic acid.

Preferred compounds of formula (I) and (II) are:

(±)-2,3,4,5-tetrahydro-7-[[[(4azabenzimidazol-2-yl)methyl]methylamino]-carbonyl]-4-(3,3-dimethylbutyl)-3-oxo-1H-1,4-benzodiazepine-2-acetic acid;

(±)-10,11-dihydro-3-[4-(pyridin-2-ylamino)-1-butyl]-5H-dibenzo[a,d]cycloheptene-10-acetic acid;

(+/−)-2,3,4,5-Tetrahydro-7-[[(benzimidazol-2-yl)thiomethyl]carbonyl]4-methyl-3-oxo-1H-1,4-benzodiazepine-2-acetic acid;

(±)-8-[2-(2-aminothiazol-4-yl)-1-ethoxy]-2-methyl-3-oxo-2,3,4,5-tetrahydro-1H-2-benzazepine-4-acetic acid;

(S)-2,3,4,5-Tetrahydro-4-methyl- 3-oxo-7-[[[(4,5,6,7-tetrahydrobenzimidazol-2-yl)methyl]methylamino]carbonyl]-1H-1,4-benzodiazepine-2-acetic acid;

(±)-8-[3-[(2-diazepin-2-yl)amino]-1-propyloxy]-2-methyl-3-oxo-2,3,4,5-tetrahydro-1H-2-benzazepine-4-acetic acid.

More preferred compounds of formula (I) and (II) are:

(S)-7-[[(2-benzimidazolylmethyl)-N-methylamino]carbonyl]-4-(4-trifluoromethoxy-benzyl)-3-oxo-2,3,4,5-tetrahydro-1H-1,4-benzodiazepine-2-acetic acid;

7-[[(1H-imidazol-2-yl)-2-amino]ethylamino]carbonyl-3-oxo-4-(2-phenylethyl)-2,3,4,5-tetraydro-1H-1,4-benzodiazepine-2-acetic acid;

(S)-3-oxo-8-[3-(pyridin-2-ylamino)-1-propyloxy]-2-(2,2,2-trifluoroethyl)-2,3,4,5-tetrahydro-1H-2-benzazepine-4-acetic acid;

(S )-8-[2-[6-(methylamino)pyridin-2-yl]-1-ethoxy]-3-oxo-2-[4-trifluoromethyl)-benzyl]-2,3,4,5-tetrahydro-1H-2-benzazepine-4-acetic acid;

(S)-8-[3-(4-methylpyridin-2-ylamino)-1-propyloxy]-3-oxo-2-[4-(trifluoromethyl)-benzyl]-2,3,4,5-tetrahydro-1H-2-benzazepine-4-acetic acid;

(S)-3-oxo-8-[3-(pyridin-2-ylamino)-1-propyloxy]-2-(2-phenylethyl)-2,3,4,5-tetrahydro-1H-2-benzazepine-4-acetic acid;

(±)-10,11-dihydro-3-[3-(3,4,5,6-tetrahydropyrinmidin-2-ylamino)-1-propyloxy]-5H-dibenzo[a,d]cycloheptene-10-acetic acid;

(S)-8-[2-[6-(methylamino)pyridin-2-yl]ethoxy]-3-oxo-2-(2-phenylethyl)-2,3,4,5-tetrahydro-1H-2-benzazepine4-acetic acid;

(±)-8-[3-[(2-imidazolin-2-yl)amino]-1-propyloxy]-2-methyl-3-oxo-2,3,4,5-tetrahydro-1H-2-benzazepine-4-acetic acid.

Other compounds that are useful in this invention are disclosed in WO 97/23451, and are described according to formula (III.). Exemplary are those

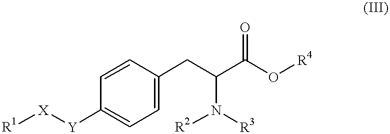

compounds wherein X represents alkylene with 1-6 C atoms or 1,4-piperidyl; Y is absent or represents O, CONH or —C═C—; R¹ represents H, CN, N₃, NH₂, H₂N—C(=NH) or H₂N—(C═NH)—NH (the primary amino groups can also be provided with conventional amino protective groups): R², R³ each independently of one another represent H, A, A—SO₂—) Ar—SO₂, campher-10—SO₂—, COOA or a conventional amino protective group; A, R⁴ each independently of one another represent H or alkyl with 1-10 C atoms or benzyl; and Ar represents phenyl or benzyl which is unsubstituted or single-substituted with CH₃. A representative compound is given by formula (IIIa):

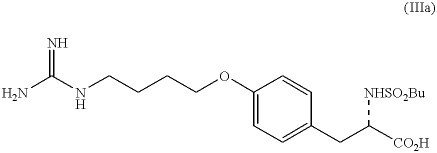

The disclosure of WO 97/23451 (PCT/EP96/05646, Merck) is incorporated herein by reference as though fully set forth.

Other compounds that are useful in this invention are disclosed in WO 97/08145,and are described according to the general formula (IV).

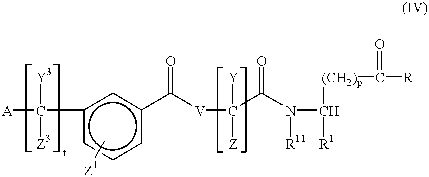

The disclosure of WO 97/08145 (PCT/US96/13500, G. D. Searle) is incorporated herein by reference as though fully set forth.

Other compounds that are useful in this invention are disclosed in WO 97/23480, and are described according to the general formula (V).

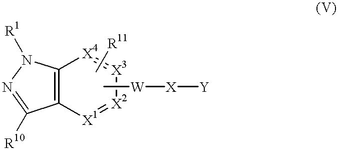

A representative compound is given by formula (Va):

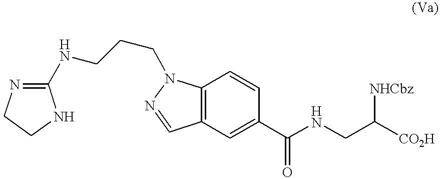

In addition, compounds of formula (V) wherein the indazole ring is replaced by a 1,4-substituted piperidinyl or piperazinyl ring are useful. A compound of formula (Vb), such as that disclosed in Bioorg. Med. Chem. Lett., 7, 1371 (1997), is representative:

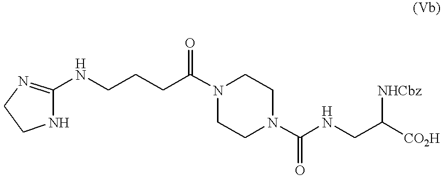

The disclosure of WO 97/23480 (PCT/US96/20523, Du Pont Merck) is incorporated herein by reference as though fully set forth.

Other compounds that are useful in this invention are disclosed in WO 96/37492, and are described according to the general formula (VI).

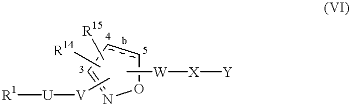

A representative compound is given by formula (VIa-c):

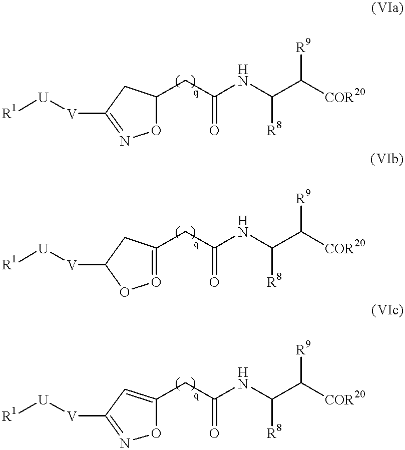

A representative compound is given by formula (Vld):

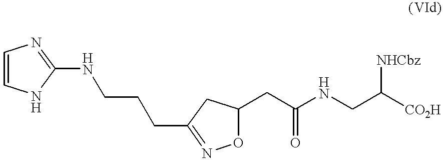

The disclosure of WO 96/37492 (PCT/US96/07646, Du Pont Merck) is incorporated herein by reference as though fully set forth.

Other compounds that are useful in this invention are disclosed in WO 95/327 10, and are described according to the general formula (VII).

X—Y—Z—Aryl—A—B (VII)

Representative compounds of this type is given by formula (VIIa-b):

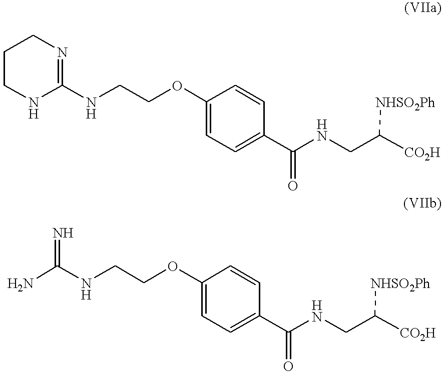

The disclosure of WO 95/32710 (PCT/US95/05938, Merck) is incorporated herein by reference as though fully set forth.

Other compounds that are useful in this invention are disclosed in WO 93/08174, and are described according to the general formula (VIII).

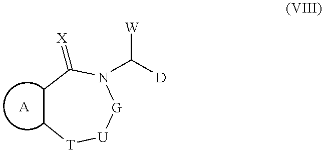

A representative compound of this type is given by formula (VIIa):

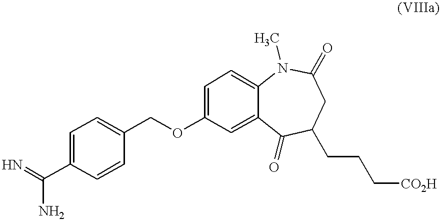

The disclosure of WO 93/08174 (PCT/US92/08788, Genentech) is incorporated herein by reference as though fully set forth.

Other compounds that are useful in this invention are disclosed in WO 95/04057, and are described according to the general formula (IX) and (X).

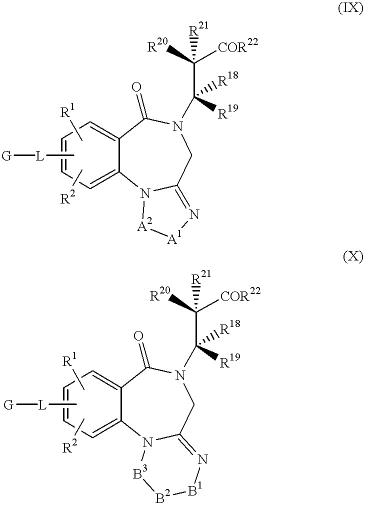

The disclosure of WO 95/04057 (PCT/US94/07989, Genentech) is incorporated herein by reference as though fully set forth.

Also included in this invention are pharmaceutically acceptable addition salts, complexes or prodrugs of the compounds of this invention. Prodrugs are considered to be any covalently bonded carriers which release the active parent drug according to formula (1) in vivo. In cases wherein the compounds of this invention may have one or more chiral centers, unless specified, this invention includes each unique nonracemic compound which may be synthesized and resolved by conventional techniques. In cases in which compounds have unsaturated carbon-carbon double bonds, both the cis (Z) and trans (E) isomers are within the scope of this invention. In cases wherein compounds may exist in tautomeric forms, such as keto-enol tautomers, such as

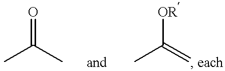

tautomeric form is contemplated as being included within this invention whether existing in equilibrium or locked in one form by appropriate substitution with R′. The meaning of any substituent at any one occurrence is independent of its meaning, or any other substituent's meaning, at any other occurrence, unless specified otherwise.

Abbreviations and symbols commonly used in the peptide and chemical arts are used herein to describe the compounds of this invention.

C₁₋₄alkyl as applied herein is meant to include methyl, ethyl, n-propyl, isopropyl. n-butyl, isobutyl and t-butyl. C₁₋₆alkyl additionally includes pentyl, n-pentyl, isopentyl. neopentyl and hexyl and the simple aliphatic isomers thereof. Any C₁₋₄alkyl or C₁₋₆alkyl group may be optionally substituted by R⁷ unless otherwise indicated. C₀₋₄alkyl and C₀₋₆alkyl additionally indicates that no alkyl group need be present (e.g., that a covalent bond is present).

C₂₋₆alkenyl as applied herein means an alkyl group of 2 to 6 carbons wherein a carbon-carbon single bond is replaced by a carbon-carbon double bond. C₂₋₆alkenyl includes ethylene, 1-propene, 2-propene, 1-butene, 2-butene, isobutene and the several isomeric pentenes and hexenes. Both cis and trans isomers are included. Any C₂₋₆alkenyl group may be optionally substituted by R⁷ unless otherwise indicated.

C₂₋₆alkynyl means an alkyl group of 2 to 6 carbons wherein one carbon-carbon single bond is replaced by a carbon-carbon triple bond. C₂₋₆alkynyl includes acetylene, 1-propyne, 2-propyne, 1-butyne, 2-butyne, 3-butyne and the simple isomers of pentyne and hexyne. Any sp³ carbon atom in the C₂₋₆alkynyl group may be optionally substituted by R⁷.

C₁₋₄oxoalkyl refers to an alkyl group of up to four carbons wherein a CH₂ group is replaced by a C(O), or carbonyl, group. Substituted formyl, acetyl, 1-propanal, 2-propanone, 3-propanal, 2-butanone, 3-butanone, 1- and 4-butanal groups are representative. C₁₋₆oxoalkyl includes additionally the higher analogues and isomers of five and six carbons substituted by a carbonyl group. C₃₋₆oxoalkenyl and C₃₋₆oxoalkynyt refers to a C₃₋₆alkenyl or C₃₋₆alkynyl group wherein a CH₂ group is replaced by C(O) group. C₃₋₄oxoalkenyl includes 1-oxo-2-propenyl, 3-oxo-l-propenyl, 2-oxo-3-butenyl and the like.

A substituent on a C₁₋₆alkyl, C₂₋₆alkenyl, C₂₋₆alkynyl or C₁₋₆oxoalkyl group, such as R⁷, may be on any carbon atom which results in a stable structure, and is available by conventional synthetic techniques.

R⁷ is independently H, halo, —OR⁸, —SR⁸, —CN, —NR′R⁸, —NO₂, —CF₃, CF₃S(O)ᵣ—, —CO₂R′, —CONR′₂, R⁹—C₀₋₆alkyl-, R⁹—C₁₋₆oxoalkyl-, R⁹—C₂₋₆alkenyl-, R⁹—C₂₋₆alkynyl-, R⁹—C₀₋₆alkyloxy-, R⁹—C₀₋₆alkylamino- or R⁹—C₀₋₆alkyl—S(O)ᵣ-; wherein R⁸ is R′, —C(O)R′, —C(O)NR′₂, —C(O)OR⁵, —S(O)ₘR′ or S(O)₂NR′₂, and R⁹ is H, C₃₋₆cycloalkyl, Het or Ar;

R⁹-C₁₋₆alkyl refers to a C_{1- 6}alkyl group wherein in any position a carbon-hydrogen bond is replaced by a carbon-R⁹ bond. R⁹-C₂₋₆alkenyl and R⁹-C₂₋₆alkynyl have a similar meaning with respect to C₂₋₆alkenyl and C₂₋₆alkynyl.

Ar, or aryl, as applied herein, means phenyl or naphthyl, or phenyl or naphthyl substituted by one to three moieties R⁷. In particular, R⁷ may be C₁₋₄alkyl, C₁₋₄alkoxy, C₁₋₄alkylthio, trifluoroalkyl, OH, F, Cl, Br or I.

Het, or heterocycle, indicates an optionally substituted five or six membered monocyclic ring, or a nine or ten-membered bicyclic ring containing one to three heteroatoms chosen from the group of nitrogen, oxygen and sulfur, which are stable and available by conventional chemical synthesis. Illustrative heterocycles are benzofuran, benzimidazole, benzopyran, benzothiophene, furan, imidazole, indole, indoline, morpholine, piperidine, piperazine, pyrrole, pyrrolidine, tetrahydropyridine, pyridine, thiazole, thiophene, quinoline, isoquinoline, and tetra- and perhydro- quinoline and isoquinoline. A six membered ring heterocycle containing one or two nitrogens, such as piperidine, piperazine, tetrahydropyridine and pyridine, are preferred heterocycles for the moiety Z. Any accessible combination of up to three substituents, such as chosen from R⁷. on the Het ring that is available by chemical synthesis and is stable is within the scope of this invention.

C₃₋₇cycloalkyl refers to an optionally substituted carbocyclic system of three to seven carbon atoms, which may contain up to two unsaturated carbon-carbon bonds. Typical of C₃₋₇cycloalkyl are cyclopropyl, cyclobutyl, cyclopentyl, cyclopentenyl, cyclohexyl, cyclohexenyl and cycloheptyl. Any combination of up to three substituents, such as chosen from R⁷, on the cycloalkyl ring that is available by conventional chemical synthesis and is stable, is within the scope of this invention.

When R^{b} and R^{c} are joined together to form a five- or six-membered aromatic or non-aromatic ring fused to the ring to which R^{b} and R^{c} are attached, the ring formed will generally be a five- or six-membered heterocycle selected from those listed above for Het, or will be a phenyl, cyclohexyl or cyclopentyl ring. Benzimidazolyl, 4-azabenzimidazolyl, 5-azabenzimidazolyl and substituted derivatives thereof are preferred moieties for W when R^{b} and R^{c} are joined together to form a ring.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical, BrZ refers to the o-bromobenzyloxycarbonyl radical, ClZ refers to the o-chlorobenzyloxycarbonyl radical, Bn refers to the benzyl radical. 4-MBzl refers to the 4-methyl benzyl radical, Me refers to methyl, Et refers to ethyl, Ac refers to acetyl, Alk refers to C₁₋₄alkyl, Nph refers to 1- or 2-naphthyl and cHex refers to cyclohexyl. MeArg is N^{α}-methyl arginine.

Certain reagents are abbreviated herein. DCC refers to dicyclohexylcarbodiimide. DMAP refers to dimethylaminopyridine, D[EA refers to diisopropylethylamine, EDC refers to N-ethyl-N′(dimethylarminopropyl)carbodiimide. HOBt refers to 1-hydroxybenzotriazole, THF refers to tetrahydrofuran. DMF refers to dimethyl formamide, NBS refers to N-bromo-succinimide, Pd/C refers to a palladium on carbon catalyst, DPPA refers to diphenylphosphoryl azide, BOP refers to benzotriazol-1-yloxy-tris (dimethylamino)phosphonium hexafluorophosphate, HF refers to hydrofluoric acid, PPA refers to polyphosphoric acid, TEA refers to triethylamine, TFA refers to trifluoroacetic acid, PCC refers to pyridinium chlorochromate.

The compounds of formula (I) and (II) may be prepared by methods common to the art, such as disclosed in the Example section herein, and published in Blackbum, et al.. WO 93108174; Bondinell, etal., WO 95/18619; Bondinell, etal., WO 94/14776; to Blackburn, et al. WO 95/04057; Ali et al., WO 96/00730; Keenan et al., WO 96/00574; Bondinell et al., WO 97/01540; Miller et al., WO 97/24119, Ali et al., WO 97/24122; and Ali et al., WO97/24124, and in references cited therein. The peptides cited herein are made by common methods such as disclosed in EP-A 0 341 915. The compounds of formula (III)-(X) may be prepared according to references cited herein as describing them.

Acid addition salts of the compounds are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts.

This invention also provides a pharmaceutical composition which comprises a compound according to formula (I)-(X) and a pharmaceutically acceptable carrier. Accordingly, the compounds of formula (I)-(X) may be used in the manufacture of a medicament. Pharmaceutical compositions of the compounds of formula (I)-(X) prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in a emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compounds of this invention may also be combined with excipients such as cocoa butter, glycerin, gelatin or polyethylene glycols and molded into a suppository.

The compound is administered either orally or parenterally to the patient, in a manner such that the concentration of drug is sufficient to promote bone formation. The pharmaceutical composition containing the peptide is administered at an oral dose of between about 0.1 to about 50 mg/kg in a manner consistent with the condition of the patient. Preferably the oral dose would be about 0.5 to about 20 mg/kg. For acute therapy, parenteral administration is preferred. An intravenous infusion of the peptide in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg. The compounds are administered one to four times daily at a level to achieve a total daily dose of about 0.4 to about 400 mg/kg/day. The precise dosage and method by which the compounds are administered is readily determined by one routinely skilled in the art by comparing the blood level of the agent to the concentration required to have a therapeutic effect.

This invention is a method for treating or preventing conditions where deficiency of mineralized bone mass creates pathology or where bone remodeling or healing is desired, which comprises administering a compound of this invention, as hereinbefore defined, especially a compound according to any one of formula (I)-(X). In particular. the administration of a compound of this invention is indicated for fracture healing and the prevention of bone fractures. Diseases and metabolic disorders which result in loss of bone structure also benefit from such treatment. For instance, hyperparathyroidism. Paget's disease, osteomalacia, hypercalcemia of malignancy, osteolytic lesions produced by bone metastasis, and bone loss due to immobilization or sex hormone deficiency. would benefit from administering a compound of this invention.

The compounds of this invention may also be used in combination with other therapies for the treatment of diseases or pathologies of the bone. For instance, one may administer the bone-forming compounds of this invention with a compound which inhibits the resorption of bone, such as an inhibitor of osteoclast function. Inhibitors of cathepsin K and vitronectin receptor antagonists are known to inhibit osteoclast function. and other such inhibitors are also known in the art. In a preferred embodiment, the compounds of this invention may both inhibit bone resorption and stimulate bone formation.

The compounds may be tested in one of several biological assays to determine the concentration of compound which is required to have a given pharmacological effect.

ROS Osteocalcin assay

Cell culture: ROS 17/2.8 osteoblasts were routinely cultured in Ham's F-12 medium containing 5% FBS, 25 rmM HEPES (pH 7.4), 1.1 mM CaCl₂, penicillin (10 U/ml) and streptomycin (10 ug/ml). The cells were subcultured twice a week using 0.04% trypsin containing 5 mM EDTA to detach the cells.

For the assay, 30,000 cells were plated per well in 24-well multidishes in 0.5 ml of HAM's F-12 medium containing 1% FBS and allowed to adhere for 3-4 hr at 37° C. in a humidified incubator with 5% CO₂. Increasing concentrations of test compounds (0.01-100 mM) were then added to the wells (duplicate cultures per concentration tested) and the incubation continued for an additional 48 hr. At the end of the incubation, cultures supernatants were removed and assayed for osteocalcin levels using a rat RIA as per the manufacturer's instructions. The lower limit of detection in this assay was 0.3 ng/ml. In every experiment, Vitamin D3 (10 nM) and the peptide cyclo[Arg-Gly-Asp-D-Phe-Val] were tested as an internal controls. Typically, this concentration of Vitamin D3 and the cyclic peptide yielded a two to three fold increase over the basal level of osteocalcin. The osteocalcin levels under unstimulated conditions ranged from 0.3-0.5 ng/ml. To correct for inter-experiment variability, the EC50 value for each test compound was normalized against a fixed value for cyclo[Arg-Gly-Asp-D-Phe-Val](0.74 uM; averaged from five experiments). Activity for each compound was expressed as the EC50 from the dose-response curve.

For the compounds of Examples 1-34 the EC50 were less than 1 uM. Preferred compounds generally promote osteocalcin release at a concentration of less than 0.5 uM. More preferred compounds promote osteocalcin release at less than 0.05 uM.

Mineralization of MC3T3 osteoblasts

Materials

MC3T3-E1 cells were obtained from ATCC. Fetal bovine serum (FBS) was purchased from Hyclone Laboratories Inc. (Logan, Utah). Long acting ascorbic was purchased from Wako Pure Chem Industries, Ltd (Japan) and β-glycerophosphate from Sigma Chemical Co. (St. Louis, Mo.). [³H]thymidine [20 Ci/mmol] was purchased from Amersham Life Sciences (Arlington Heights, Ill.) and, ⁴⁵CaCl₂ purchased from. NEN Research Products (Boston, Mass.). Mouse osteocalcin RIA was obtained from British Biotechnology Ltd (Stoughton, Mass.).

Cell culture

Stock cultures of MC3T3-E1 osteoblasts were routinely maintained in Earle's minimal essential medium (GIBCO) containing 10% (v/v) fetal bovine serum, 25 mM HEPES pH 7.4, penicillin (100 ug/ml) and streptomycin (100 ug/ml). Until the time of study, cells were subcultured twice a week using 0.04% trypsin (wt/vol) with 5 mM EDTA to achieve cell detachment.

To characterize the temporal sequence of osteoblast differentiation, 30,000 cells were plated/well in 24-well multidishes and grown for periods ranging from 3-26 days in a-MEM medium (GIBCO) containing 10% fetal bovine serum (V/V), penicillin (100 U/ml) and streptomycin (100 ug/ml), with or without supplementation with 5 mM β-glycerophosphate and 100 uM ascorbic acid in the presence or absence of RGD compounds. (No Alzirin red staining was observed for cultures which are not treated with β-glycerophosphate and ascorbic acid.) Media were removed and replaced with fresh media containing supplements every 4-5 days.

Assay of cell replication

At the completion of the incubation period, cells were harvested by removing the media, washing twice with PBS and treated with 0.04% trypsin containing 5 mM EDTA to detach the cells. Cell number was determined at the various time points by direct counting with a hemocytometer. DNA synthesis was measured by determining TCA-precipitable radioactivity following a 4 hour pulse with [³H]thymidine (1.5 uCi/ml).

⁴⁵Ca Uptake

Mineralization of cells was assessed by calcium isotope accumulation. Cells were incubated for 48 h in medium containing 0.5 uCi/mi of ⁴⁵CaCl₂ at various time points after seeding. Subsequently, the monolayers were washed twice with PBS using 1 ml/wash. Cells were harvested and digested in 0.1 N NaOH and aliquots were counted by liquid scintillation counting using a Beckman 5500 scintillation counter.

Alizarin-Red Staining

Calcified nodules in actively mineralizing cultures were visualized by staining cell monolayers with Alizarin-Red-S (Sigma Chemical Co, St Louis, Mo.). Cell cultures were washed twice with PBS, fixed for 10 min in 50 percent ethanol, rehydrated with 1 ml of distilled water for 5 min and then stained for 1-3 min with 200 uL of 1 percent Alizarin Red S (aqueous). The monolayers were then washed extensively and very carefully using 1 ml H₂O per wash. Calcified nodules which appeared a bright red color were identified by light microscopy.

Alkaline Phosphatase activity

To monitor the differentiation of cells to the mature osteoblast type, alkaline phosphatase activity was determined in cell lysates using the colorimetric assay. Cells were washed twice with PBS using 1 ml/wash. Subsequently, the monolayers were transferred to ice and cells lysed in 0.2 ml of 0.1% TX-100. The assay mixture contained 20 uL of the cell lysate (0.2-0.7 mg protein) and 80 uL of reaction buffer containing 0.1 M 2-amino-2-methyl-1-propanol, 2 mM MgCl₂, 2 mM Na₂p-nitro-phenylphosphate (Na₂PNPP). The reactions were stopped with the addition of 100 uL of ice cold 0.25 N NaOH to each reaction and absorbance was read at 410 nm. Standard curves were prepared with known amounts of p-nitrophenol. Alkaline phosphatase activity was calculated as umol of substrate (p-nitrophenol) released/mg protein/min.

Osteocalcin assay

Osteocalcin levels in the supernatants were determined at various time points during the assay using a mouse RIA as per the manufacturers instructions. The lower limits of detection in this assay was 1.5 ng/ml.

Cyclo[Arg-Gly-Asp-D-Phe-Val], which caused osteocalcin release in the ROS Osteocalcin Assay with an EC50 of 0.74 uM, was evaluated in the above mineralization assay at a concentration of 25 uM. It showed calcification nodules upon Alzirin staining, increased calcium uptake, and enhanced alkaline phosphatase activity, which are all indicators of mineralization and bone-forming activity.

Similar procedures may be applied to other osteoblastic cell lines to determine the ability of the compounds of this invention to cause mineralization and bone formation in other mammalian species and in cell lines derived from different sources.

Inhibition of Vitronectin Binding

Solid-Phase [³H]-SK&F-107260 Binding to αᵥβ₃: Human placenta or human platelet αᵥβ₃ (0.1-0.3 mg/mL) in buffer T (containing 2 mM CaCl₂ and 1% octylglucoside) was diluted with buffer T containing 1 mM CaCl₂, 1 mM MnCl₂, 1 mM MgCl₂ (buffer A) and 0.05% NaN₃, and then immediately added to 96-well ELISA plates (Coming, New York, N.Y.) at 0.1 mL per well. 0.1-0.2 μg of αᵥβ₃ was added per well. The plates were incubated overnight at 4° C. At the time of the experiment, the wells were washed once with buffer A and were incubated with 0.1 mL of 3.5% bovine serum albumin in the same buffer for 1 hr at room temperature. Following incubation the wells were aspirated completely and washed twice with 0.2 mL buffer A.

Compounds were dissolved in 100% DMSO to give a 2 mM stock solution, which was diluted with binding buffer (15 mM Tris-HCl (pH 7.4), 100 mM NaCl, 1 ,mM CaCl₂, 1 mM MnCl₂, 1 mM MgCl₂) to a final compound concentration of 100 μM. This solution is then diluted to the required final compound concentration. Various concentrations of unlabeled antagonists (0.001-100 μM) were added to the wells in triplicates, followed by the addition of 5.0 nM of [³H]-SK&F-107260 (65-86 Ci/mmol).

The plates were incubated for 1 hr at room temperature. Following incubation the wells were aspirated completely and washed once with 0.2 mL of ice cold buffer A in a well-to-well fashion. The receptors were solubilized with 0.1 mL of 1% SDS and the bound [³H]-SK&F-107260 was determined by liquid scintillation counting with the addition of 3 mL Ready Safe in a Beckman LS Liquid Scintillation Counter, with 40% efficiency. Nonspecific binding of [³H]-SK&F-107260 was determined in the presence of 2 μM SK&F-107260 and was consistently less than 1% of total radioligand input. The IC₅₀ (concentration of the antagonist to inhibit 50% binding of [³H]-SK&F- 107260) was determined by a nonlinear, least squares curve-fitting routine, which was modified from the LUNDON-2 program. The Kᵢ (dissociation constant of the antagonist) was calculated according to the equation: Kᵢ=IC₅₀/(1+L/K_{d}), where L and K_{d} were the concentration and the dissociation constant of [³H]-SK&F- 107260, respectively.

The compounds of Examples 1-34 generally inhibit vitronectin binding to SK&F 107260 at less than 1 micromolar.

Compounds of this invention are also tested for in vivo bone formation in other standard assays for evaluating bone formation, such as the ovarectornized rat model, described by Wronski et al., Cells and Materials 1991, Sup. 1, 69-74.

### General

Nuclear magnetic resonance spectra were recorded at either 250 or 400 MHz using respectively, a Bruker AM 250 or Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (δ) downfield from the internal standard tetramethylsilane. Abbreviations for NMR data are as follows: s=singlet, d=doublet, t=triplet, q=quartet, m=multiplet, dd=doublet of doublets, dt=doublet of triplets. app=apparent, br=broad. J indicates the NMR coupling constant measured in Hertz. Continuous wave infrared (IR) spectra were recorded on a Perkin-Elmer 683 infrared spectrometer, and Fourier transform infrared (FTIR) spectra were recorded on a Nicolet Impact 400 D infrared spectrometer. IR and FTIR spectra were recorded In transmission mode, and band positions are reported in inverse wavenumbers (cm-⁻¹). Mass spectra were taken on either VG 70 FE, PE Syx API III, or VG ZAB HF instruments, using fast atom bombardment (FAB) or electrospray (ES) ionization techniques. Elemental analyses were obtained using a Perkin-Eimer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel. Analytical and preparative HPLC were carried out on Rainin or Beckman chromatographs. ODS refers to an octadecylsilyl derivatized silica gel chromatographic support. 5 μ Apex-ODS indicates an octadecylsilyl derivatized silica gel chromatographic support having a nominal particle size of 5 μ, made by Jones Chromatography, Littleton, Colo. YMC ODS-AQ® is an ODS chromatographic support and is a registered trademark of YMC Co. Ltd., Kyoto, Japan. PRP-1® is a polymeric (styrene-divinylbenzene) chromatographic support, and is a registered trademark of Hamilton Co., Reno, Nev.) CELITE® is a filter aid composed of acid-washed diatomaceous silica, and is a registered trademark of Manville Corp., Denver, Colo.

Methyl (±)-7-carboxy-4-methyl-3-oxo-2,3,4,5-tetrahydro-1H-1,4-benzodiazepine-2-acetate, methyl (2S)-7-carboxy-4-methyl-3-oxo-2,3,4,5-tetrahydro-1H-1,4-benzodiazepine-2-acetate, methyl (2R)-7-carboxy-4-methyl-3-oxo-2,3,4,5-tetrahydro-1H-1,4-benzodiazepine-2-acetate, methyl (±)-7-carboxy-4-isopropyl-3-oxo-2,3,4,5-tetrahydro-1H-1,4-benzodiazepine-2-acetate, methyl (±)-7-carboxy-3-oxo-2-(2-phenylethyl)-2,3,4,5-tetrahydro-1H-1,4-benzodiazepine-2-acetate, and methyl (±)-8-carboxy-2-methyl-3-oxo-2,3,4,5-tetrahydro-1H-2-benzazepine-4-acetate were prepared by the method of Bondinell. et al., WO 93/00095. 2-(Aminomethyl)imidazole was prepared according to the procedure in Annalen 1968, 718, 249.

All of the references cited throughout the instant specification are incorporated by reference as though fully set forth herein.

### Preparation I

### Preparation of methyl (±)-7-carboxy-4-(2-methoxyethyl)-3-oxo-2,3,4,5-tetrahydro-1H-1,4-benzodiazepine-2-acetate

a) tert-Butyl 3-[(2-methoxyethyl)amino]methyl-4-nitrobenzoate

A mixture of tert-butyl 3-methyl-4-nitrobenzoate (WO 93/00095; 14-96 g, 63.05 mmol), NBS (16.83 g, 94.58 mmol), benzoyl peroxide (1.53 g, 6.31 mmol), and CCl₄ (315 mL) was heated at reflux. After 18.5 h, the reaction was cooled thoroughly in ice and filtered to remove the precipitated succinimide. The filtrate was concentrated to leave a yellow oil.

This yellow oil was dissolved in dry THF (315 mL), and 2-methoxyethylamine (16.4 mL, 189.2 mmol) was added all at once. The orangish-yellow solution was stirred at RT for 40 min, then was concentrated to remove the THF. The residue was diluted with Et₂O (630 mL) and washed sequentially with 1.0 N NaOH (125 mL) and H₂O (125 mL). The combined aqueous layers were back-extracted with Et₂O (300 mL), and the combined organic layers were washed with brine (125 mL) and dried (MgSO₄). Concentration and silica gel chromatography (3:2 EtOAc/hexanes) gave the title compound (10.30 g, 53%) as a yellow oil:

b) tert-Butyl 3-[[N-(2-methoxyethyl)-N-(tert-butoxycarbonyl)]amino]methyl-4-nitrobenzoate

Di-tert-butyl dicarbonate (7.97 g, 36.51 mmol) was added all at once to a solution of tert-butyl 3-[(2-methoxyethyl)amino]methyl-4-nitrobenzoate (10.30 g 33.19 mmol) in CHCl₃ (165 mL) at RT. After 16 h, the reaction was concentrated and reconcentrated from hexanes (to remove CHCl₃). Silica gel chromatography (20% EtOAc/hexanes) gave the title compound (13.21 g, 97%) as a yellow oil.

c) tert-Butyl 4-amino-3-[[N-(2-methoxyethyl)-N-(tert-butoxycarbonyl)]amino methyl benzoate

10% Pd/C (3.42 g, 3.22 mmol) was added to a solution of tert-butyl 3-[[N-(2-methoxyethyl) -N-(tert-butoxycarbonyl)]amino]methyl4-nitrobenzoate (13.21 g, 32.18 mmol) in EtOAc (320 mL), and the mixture was shaken on a Parr apparatus at RT under H₂ (55 psi). After 4 h, the reaction was filtered through CELITE®, and the filtrate was concentrated to afford the title compound (12.16 g, 99%) as a colorless foam.

d) t-Butyl (±)-4-[2-(1,4-dimethoxy-1,4-dioxobutyl)amino]-3-[[N-(2-methoxyethyl)-N-(tert-butoxycarbonyl)]amino]methylbenzoate

A solution of tert-butyl 4-amino-3-[[N-(2-methoxyethyl)-N-(tert-butoxycarbonyl)]amino]methylbenzoate (12.16 g, 31.96 mmol) and dimethylacetylene dicarboxylate (4.3 mL, 35.2 mmol) in MeOH (65 mL) was heated at reflux for 45 min, then was cooled to RT. The resulting solution was combined with MeOH (260 mL) and 10% Pd/C (6.80 g, 6.4 mmol), and the mixture was shaken on a Parr apparatus at RT under H₂ (50 psi). After 6.5 h, the reaction was filtered through CELITE®. and the filtrate was concentrated on the rotavap. The residue was reconcentrated from CHCl₃ (to remove MeOH), then was chromatographed on silica gel (30% EtOAc/hexanes). The title compound (15.03 g, 90%) was obtained as a faintly yellow oil.

e) Methyl (±)-7-carboxy-4-(2-methoxyethyl)-3-oxo-2,3,4,5-tetrahydro-1H-1,4-benzodiazepine-2-acetic acid

TFA (140 mL) was added all at once to a solution of t-butyl (±)-4-[2-(1,4-dimethoxy-1,4-dioxobutyl)amino]-3-[[N-(2-methoxyethyl)-N-(tert-butoxycarbonyl)]amino]methylbenzoate (15.03 g, 28.65 mmol) in anhydrous CH₂Cl₂ (140 mL) at 0° C., and the faintly yellow solution was warmed to RT. After 2 h, the solution was concentrated on the rotavap, and the residue was reconcentrated from toluene (to remove residual TFA). The resulting oil was combined with toluene (280 mL) and Et₃N (20 mL. 143 mmol), and the mixture was heated to reflux. A light yellow, homogeneous solution was produced. After 23.5 h, the reaction was concentrated on the rotavap to leave a solid residue. This was dissolved in a minimum of MeOH (ca. 720 mL) at reflux, diluted with H₂O (720 mL), and acidified with glacial AcOH (8 mL). The solution was cooled to RT. then was cooled in the refrigerator. After several h, more glacial AcOH (24 mL) was added. The mixture was kept in the refrigerator overnight then was filtered. The solid was washed sequentially with MeOH and Et₂O, then was dried in high vacuum to afford the title compound (6.40 g, 66%) as a nearly colorless powder. The mother liquors were concentrated on the rotavap to ca. 500 mL, cooled, and filtered to afford additional title compound (1.51 g, total=7.91 g, 82%) as a light yellow solid: mp 226-229.5° C.

### Preparation 2

Using the procedures of Preparation 1, except substituting 3,4-methylenedioxyphenethylamine for 2-methoxyethylamine, the following compound was prepared:

a) Methyl (±)-7-carboxy-4-[2-(3,4-methylenedioxyphenyl)ethyl]-3-oxo-2,3,4,5-tetrahydro-1H-1,4-benzodiazepine-2-acetate.

### Preparation 3

Preparation of methyl (±)-7-carboxy-3-oxo-2,3,4,5-tetrahydro-1H-1,4-benzodiazepine-2-acetate

a) tert-Butyl 3-[[bis-(t-butoxycarbonyl)]amino]methyl-4-nitrobenzoate

Di-tert-butyliminodicarboxylate (4.35 g, 20.0 mmol) was added to a suspension of sodium hydride (0.48 g, 20.0 mmol) in anhydrous DMF (30 mL) at RT. After 30 minutes, a solution of t-butyl 3-bromomethyl-4-nitrobenzoate (6.3 g, 20 mmol) in DMF (15 mL) was added rapidly dropwise. After 16 h, the solvent was evaporated and the residue partitioned between EtOAc (200 mL) and water (40 mL). The organic layer was extracted with water (3×50 mL) and brine (40 mL) and dried finally over Na₂SO₄. Removal of solvent gave the crude product which was purified using flash chromatography (15:85; EtOAc:Hexane) to give the title compound (81.5%).

b) tert-Butyl 4-amino-3-[[bis-(t-butoxycarbonyl)]amino]methylbenzoate

A solution of tert-butyl 3-[[bis-(t-butoxycarbonyl)]amino]methyl4-nitrobenzoate (4.2 g, 9.3 mmol) in ethanol (150 mL) was hydrogenated at 40 psi in the presence of 10% Pd on C (0.40 g). After 30 minutes, catalyst was filtered and solvent removed to give the title compound in essentially quantitative yield.

c) (E/Z) tert-Butyl 4-[2-(1,4-dimethoxy-1,4-dioxo-2-butenyl)amino]-3-[[bis-(t-butoxycarbonyl)]amino]methylbenzoate

A solution of tert-butyl 4-amino-3-[[bis-(t-butoxycarbonyl)]amino]methyl benzoate (3.9 g, 9.2 mmol) and dimethylacetylene dicarboxylate (1.34 g, 9.4 mmol) was refluxed 1 h and evaporated to dryness to give the title compound.

d) tert-Butyl (±)4-[2-(1,4-dimethoxy-1,4-dioxobutyl)amino]-3-[[bis-(t-butoxycarbonyl)]amino]methylbenzoate

A solution of (E/Z) tert-butyl 4-(2-(1,4-dimethoxy-1,4-dioxo-2-butenyl)amino]-3-[[bis-(t-butoxycarbonyl)]amino]methylbenzoate (5.2 g, 9.2 mmol) in methanol ( 150 mL) was hydrogenated at 40 psi in the presence of 10% Pd/C (0.75 g). After 2 h, the catalyst was removed by filtration, and the solvent was removed to provide the crude product. Purification by flash chromatography gave the title compound (80%).

e) (±)4-[2-(1,4-Dimethoxy-1,4-dioxobutyl)amino]-3-(aminomethyl)benzoic acid, bis-(trifluoroacetate)

A solution of tert-butyl 4-[2-(1,4-dimethoxy-1,4-dioxobutyl)amino]-3-[[bis-(t-butoxycarbonyl)]amino]methylbenzoate (4.0 g, 7.1 mmol) in a mixture of methylene chloride (100 mL) and trifluoroacetic acid (25 mL) was kept 16 h at RT. The solvents were evaporated and the residue was triturated with ether to give the title compound in essentially quantitative yield.

f) Methyl (±)-7-carboxy-3-oxo-2,3,4,5-tetrahydro-1H-1,4-benzodiazepine-2-acetate

A solution of sodium methoxide in methanol (25 wt %, 6.7 mL, 30 mmol) was added to a solution of 4-[2-(1,4-dimethoxy-1,4-dioxobutyl)amino]-3-(aminomethyl) benzoic acid, bis-(trifluoroacetate) (4.0 g, 7.0 mmol) at −10° C. under argon. After 30 minutes, the cold solution was quenched with acetic acid (1.5 ml). The reaction mixture was kept one h at −20° C. and filtered. The filter cake was slurried in water (30 mL) and filtered to provide the title compound (65%).

### Preparation 4

Preparation of N-(2-Pyridinyl)ethylenediamine

a) N-Acetyl-N′-(1-oxo-2-pyridinyl)ethylenediaamine

A mixture of N-(acetyl)ethylenediamine (0.5 g, 5 mmol), 2-chloropyridine-N-oxide hydrochloride (1.6 g, 10 mmol), NAHCO₃ (1.6 g, 19 mmol), and n-butanol (5 mL) was heated to 100° C. for 18 h. The mixture was then allowed to cool, and it was filtered and the filtrate concentrated. The resulting residue was purified by chromatography (silica gel, step gradient, 2%-10% CH₃OH/CH₂Cl₂) to give the title compound as a pale yellow solid (0.40 g, 45%).

b) N-(1-oxo-2-pyridinyl)ethylenediamine

A mixture of the compound of Preparation 4(a)(0.4.g) and concentrated HCl (50 mL was heated to 90° C. for 4 d, concentrated, and the residue was recrystallized (CH₃OH:CHCl₃) to give the title compound as off-white needles (3.2 g, 82%).

c) N-(2-Pyridinyl)ethylenediamine

A mixture of the compound of Preparation 4(b)( 1.0 g, 5 mmol), potassium formate (2.2 g, 25 mmol), 10% Pd/C (0.30 g), and CH₃OH (20 mL) was heated to reflux under argon for 48 h. The mixture was filtered, the filtrate was concentrated, and the residue was purified by chromatography (silica gel, 10% CH₃OH/CH₂Cl₂) to give the title compound as an amber oil (0.30 g, 41%).

### Preparation 5

Preparation of methyl (±)-8-hydroxy-3-oxo-2,3,4,5-tetrahydro-1H-2-benzazeoine-4-acetate

a) 4-Bromo-3-bromomethylanisole

A mixture of 2-bromo-5-methoxytoluene (20 g, 0.10 mol), N-bromosuccinimide (19.6 g, 0.11 mol), benzoyl peroxide (1 g, 4 mmol), and methylene chloride (200 mL) was irradiated for 18 hr with a flood lamp to effect gentle reflux. The mixture was then cooled to −10° C. for several hours and the solution was decanted away from the precipitated succinimide. The solution was concentrated and the residue was crystallized from chloroform/hexane to give the title compound (19.7 g, 70%) as pale yellow prisms.

b) 3-Bis(tert-butoxycarbonyl)aminomethyl-4-bromoanisole

A mixture of 4-bromo-3-bromomethylanisole (24 g, 86 mmol) and potassium di-tert-butyl iminodicarboxylate (24 g, 94 mmol) in dimethylformamide (200 mL) was stirred under argon at room temperature for 18 hr. The reaction was then concentrated under vacuum and the residue was partitioned between ethyl acetate and water. The organic phase was washed with water and brine, dried(MgSO₄), and concentrated. The residue was recrystallized from hexane to give the title compound (15 g, 42%) as a white solid.

c) Methyl (±)-3-carbomethoxy-4-[2-bis(tert-butoxycarbonyl)aminomethyl-4-methoxyphenyl]-3-butenoate

A 500 mL flask was charged with 3-bis(tert-butoxycarbonyl)aminomethyl-4-bromoanisole (15 g, 36 mmol), dimethyl itaconate (7.5 g, 47 mmol), tri-o-tolylphosphine (1 g, 3 mol), palladium acetate (0.4 g, 2 mmol), diisopropylethylamine (12.8 mL, 72 mmol), and propionitrile (150 mL). The mixture was purged with argon (several evacuation/argon flush cycles), then was heated to reflux under argon for 1 hr. The reaction was allowed to cool to RT, then was poured into ice-cold ethyl ether (500 mL). The resulting precipitate was removed by filtration and the filtrate was concentrated. The residue was purified by chromatography on silica gel (10% -20% ethyl acetate in hexane) to give the title compound (11.8 g, 66%) as a pale yellow oil.

d) Methyl (±)-3-carbomethoxy-4-[2-bis(tert-butoxycarbonyl)aminomethyl-4-methoxyphenyl]butanoate

A pressure vessel charged with methyl (±)-3-carbomethoxy-4-[2-bis(tert-butoxycarbonyl)aminomethyl-4-methoxyphenyl]-3-butenoate ( 11.8 g), ethyl acetate (120 mL), and 10% palladium on charcoal (1 g) was shaken under 45 psi of hydrogen for 18 hr. The mixture was then filtered and the filtrate was concentrated to give the title compound (12 g, 100%) as a colorless oil.

e) Methyl (±)-3-carbomethoxy-4-[2-(aminomethyl)-4-methoxyphenyl]butanoate

A solution of methyl (±)-3-carbomethoxy-4-[2-bis(tert-butoxycarbonyl)aminomethyl-4-methoxyphenyl]butanoate (12 g) in chloroform (100 mL) and trifluoroacetic acid (50 mL) was stirred under argon at room temperature for 4 hr. The solution was then concentrated under vacuum to give the title compound (10 g, 100%) as a viscous oil.

f) Methyl (±)-8-methoxy-3-oxo-2,3,4,5-tetrahydro-1H-2-benzazepine-4-acetate

A solution of methyl (±)-3-carbomethoxy-4-[2-(aminomethyl)4-methoxyphenyl]butanoate (10 g, 24 mmol) and triethylamine (17 mL, 120 mmol) in toluene (100 mL) was heated at reflux for 18 hr. The reaction was then concentrated and the residue was partitioned between ethyl acetate and water. The aqueous layer was extracted twice with ethyl acetate and the combined organic extracts were washed with brine, dried (MgSO₄), and concentrated to afford the title compound (4.8 g, 76%) as tan solid.

g) Methyl (±)-8-hydroxy-3-oxo-2,3,4,5-tetrahydro-1H-2-benzazepine-4-acetate

Anhydrous aluminum chloride (7.6 g, 57 mmol) was added portionwise to a stirred solution of methyl (±)-8-methoxy-3-oxo-2,3,4,5-tetrahydro- 1H-2-benzazepine4-acetate (3.0 g, 11 mmol) and ethanethiol (4.2 mL, 57 mmol) in methylene chloride (100 mL) at 0° C. under argon. The resulting mixture was allowed to warm to room temperature and stirred overnight, then was concentrated. The residue was triturated with ice-water, and the resulting solid was collected by filtration and dried to give the title compound (2.64 g, 91%) as an off-white solid.

### Preparation 6

Preparation of methyl (±)-8-hydroxy-2-methyl-3-oxo-2,3,4,5-tetrahydro-1H-2-benzazepine-4-acetate

a) 3-(N-(tert-Butoxycarbonyl)-N-methylamino]methyl-4-bromoanisole 40% aqueous methylamine (49 mL, 563 mmole) was added rapidly to a solution of 4-bromo-3-bromomethylanisole (15.76 g, 56.29 mmole) in THF (280 mL) at RT. After 2.5 hr, the reaction was concentrated, and the residue was partitioned between Et₂O (560 mL) and 1.0 N NaOH (100 mL). The layers were separated, and the organic layer was dried (MgSO₄) and concentrated to a yellow oil.

The oil was dissolved in CHCl₃ (280 mL), and di-tert-butyl dicarbonate (1.29 g. 56.29 mmole) was added. The reaction was stirred at RT for 45 min, then was concentrated. Silica gel chromatography (5% EtOAc/toluene) gave the title compound (16.81 g, 90%) as a light yellow oil.

b) Methyl (±)-3-carbomethoxy-4-[2-N-(tert-butoxycarbonyl)-N-methylamino]methyl-4-methoxyphenyl]butanoate

A solution of 3-[N-(tert-butoxycarbonyl)-N-methylamino]methyl-4-bromoanisole (4.95 g, 15 mmol), dimethyl itaconate (3.08 g, 19.5 mmol), palladium acetate (168 mg, 0.75 mmol), tri-o-tolylphosphine (457 mg, 1.5 mol), and diisopropylethylamine (5.2 mL, 30 mmol) in propionitrile (75 mL) was heated to reflux for 45 min, then was concentrated on the rotavap. The residue was diluted with Et₂O (150 mL), and the mixture was filtered through CELITEO® to remove insoluble materials. The filtrate was concentrated, and the residue was reconcentrated from xylenes. Chromatography on silica gel (gradient: 20% EtOAc/hexanes, then 1:1 EtOAc/hexanes) removed the phosphine and baseline materials; all other materials with R_{f} 0.40-0.70 were collected together and concentrated to leave a cloudy, yellow oil.

The oil was dissolved in MeOH (75 mL), and 10% Pd/C was added carefully. The mixture was shaken under hydrogen (50 psi) for 2.5 hr, then was filtered through CELITE® to remove the catalyst. The filtrate was concentrated, and the residue was resubmitted to the reaction conditions. After another 2.5 hr, the mixture was filtered through CELITE® to remove the catalyst, and the filtrate was concentrated to leave a light yellow oil. This was reconcentrated from CHCl₃/hexanes, then was chromatographed on silica gel (gradient: 20% EtOAc/hexanes, then 1:1 EtOAc/hexanes) to afford the title compound (4.53 g, 74%) as a light yellow oil.

c) Methyl (±)-3-carbomethoxy4-[2-(methylamino)methyl-4-methoxyphenyl butanoate TFA (55 mL) was added all at once to a solution of methyl (±)-3-carbomethoxy-4-[2-[N-(tert-butoxycarbonyl)-N-methylamino]methyl-4-methoxyphenyl]butanoate (4.53 g 11.06 mmole) in anhydrous CH₂Cl₂ (55 mL) at 0° C., and the reaction was warmed to RT. After 1 hr, the reaction was concentrated, and the residue was reconcentrated from toluene (2×100 mL) to leave the title compound (11.06 mmole, quantitative) as a light yellow oil.

d) Methyl (±)-8-methoxy-2-methyl-3-oxo-2,3,4,5-tetrahydro-1H-2-benzazepine-4-acetate

A solution of methyl (±)-3-carbomethoxy-4-[2-(methylaminomethyl-4-methoxyphenyl]butanoate (11.06 mmole) and diisopropylethylamine (5.8 mL, 33.18 mmole) in toluene (110 mL) was heated at reflux for 25 hr, stirred at RT for 4 days, then heated at reflux for another 24 hr. Concentration and silica gel chromatography (5% MeOH in 1:1 EtOAc/CHCl₃) gave the title compound (2.88 g, 94%) as a light yellow solid.

e) Methyl (±)-8-hydroxy-2-methyl-3-oxo-2,3,4,5-tetrahydro-1H-2-benzazepine-4-acetate

Anhydrous aluminum chloride (1.35 g, 10.15 mmole) was added all at once to a solution of methyl (±)-8-methoxy-2-methyl-3-oxo-2,3,4,5-tetrahydro-1H-2-benzazepine-4-acetate (562 mg, 2.03 mmole) and ethanethiol (0.75 mL, 10.15 mmole) in anhydrous CH₂Cl₂ (20 mL) at 0° C. under argon. The mixture was warmed to RT and stirred for 4.5 hr, then was recooled to 0° C. Ice cold H₂O (20 mL) was added, and the mixture was stirred briskly for 5 min, then was extracted with CHCl₃ (3×20 mL). The combined CHCl₃ layers were dried (MgSO₄) and concentrated to leave a residue. The aqueous layer was suction filtered to collect a solid precipitate. This precipitate and the residue from the CHCl₃ layer were combined in 1:1 MeOH/CHCl₃, and the solution was concentrated to leave an off-white solid. This was triturated with hot MeOH, and the mixture was allowed to cool to RT. The solid was collected by suction filtration and washed sequentially with cold MeOH and Et₂O. Drying in high vacuum at 40° C. gave the title compound (467.9 mg, 88%) as a colorless solid.

### Preparation 7

Preparation of methyl (S)-8-hydroxy-3-oxo-2-(2-phenylethyl)-2,3,4,5-tetrahydro-1H-2-benzazepine-4-acetate

a) 3-[N-(tert-Butoxycarbonyl)-N-(2-phenylethyl)amino]methyl-4-bromoanisole 2-Phenethylamine (19.0 mL, 150 mmole) was added all at once to a solution of 4-bromo-3-bromomethylanisole (14.0 g, 50.0 mmole) in anhydrous THF (200 mL) at RT. After 18 hr the mixture was concentrated. The residue was dissolved in 2 M NaOH (300 mL) and extracted with CH₂Cl₂ (3×200 mL). The combined CH₂Cl₂ layers were dried over MgSO₄ and concentrated. The crude material was filtered through a plug of silica gel using 50% EtOAc/hexanes as eluent. The filtrate was concentrated under reduced pressure to give a yellow oil.

The above yellow oil was dissolved in anhydrous THF (200 mL) and di-tert-butyl dicarbonate (13.0 g, 60.0 mmole) was added all at once at RT. After 1 hr the solution was concentrated. Flash silica gel chromatography (10% EtOAc/hexanes) gave the title compound as an off-white solid (20.8 g, 100% from 4-bromo-3-bromomethylanisole).

b) 4-[2-[N-(tert-Butoxycarbonyl)-N-(2-phenylethyl)amino]methyl-4-methoxyphenyl]propionic acid

A solution of 3-[N-(tert-butoxycarbonyl)-N-(2-phenylethyl)amino]methyl-4-bromoanisole (20.0 g, 48.0 mmole), benzyl acrylate (23.0 g, 144 mmole), palladium acetate (540 mg, 2.40 mmole), tri-o-tolylphosphine (1.46 g, 4.80 mmole), and diisopropylethylamine (17.0 mL, 96.0 mmole) in propionitrile (250 mL) was deoxygenated (3x evacuation/argon purge cycles), then was heated to reflux under argon. After 48 hr the reaction was cooled to RT, filtered through a pad of CELITE®, and concentrated. Flash silica gel chromatography (10% EtOAc/hexane) gave a yellow oil, which was dissolved in 10% EtOAc/hexanes (100 mL) and left at 4° C. for 72 hr. The yellow precipitate was removed by filtration then the solution was concentrated to give a faint yellow oil (14.98 g, 62%).

The above oil was dissolved in MeOH (150 mL) and 10% Pd/C (6.40 g 6.00 mmole) was added at 0° C. The mixture was warmed to RT, shaken under hydrogen (50 psi) for 7 hr, then was filtered through a pad of CELITE® to remove the catalyst. The filtrate was concentrated under reduced pressure to give the title compound as a thick yellow oil (10.35 g, 83%).

c) (R)-1,1-Dimethylethyl[[5methoxy-2-3-oxo-3-[2-oxo-4-(phenylmethyl)-3-oxazolidinyl]propyl]phenyl]methyl](2-phenylethyl)carbonate

To a solution of 4-[2-[N-(tert-butoxycarbonyl)-N-(2-phenylethyl)amino)methyl-4-methoxyphenyl]propionic acid (10.35 g, 25.0 mmole) in CH₂Cl₂ (125 mL) was added pyridine (2.4 mL, 30.0 mmole) then cyanuric fluoride (1.4 mL, 15.0 mmole) at RT. After 2 hr the mixture was filtered through a pad of CELITE®, washed with cold H₂O (100 mL) then with brine (100 mL), dried over MgSO₄, and concentrated.

To a solution of (R)4-benzyl-2-oxazolidinone (5.30 g, 30.0 mmole) in anhydrous THF (125 mL) was added n-BuLi (11.0 mL, 2.5 M solution in hexanes, 27.5 mmole) at −78° C. After 15 minutes the above acid fluoride in anhydrous THF (25 mL) was added dropwise over 5 minutes. After 1 hr the mixture was poured into 300 mL H₂O and extracted with EtOAc (3×200 mL). The combined EtOAc layers were dried over MgSO₄ and concentrated. Flash silica gel chromatography (30% EtOAc/hexanes) gave the title compound as a thick oil (12.12 g, 85%).

d) [R-(R*, S*)]-Methyl β-[[4-methoxy-2-[[[(1,1-dimethylethoxy)carbonyl](2-phenylethyl)amino]methyl]phenyl]methyl]-γ-oxo-4-(phenylmethyl)-3-oxazolidinebutanoate

To a solution of (R)-1,1-dimethylethyl[[5-methoxy-2-[3-oxo-3-[2-oxo4-(phenylmethyl)-3-oxazolidinyl]propyl]phenyl]methyl](2-phenylethyl)carbamate (12.12 g, 21.0 mmole) in anhydrous THF (100 mL) was added lithium bis(crimethylsilyl)amide (22.0 mL, 1M in THF, 22.0 mmole) at −78° C. After 15 minutes methyl bromoacetate (9.9 mL, 105 mmole) was added then the mixture was warmed to −20° C. After 3 hr the mixture was poured into 200 mL H₂O and extracted with EtOAc (3×500 mL). The combined EtOAc layers were dried over MgSO₄ and concentrated. Flash silica gel chromatography (25% EtOAc/hexanes) gave 9.91 g of a 3:2 mixture (HPLC. 20% EtOAc/hexanes) of the title compound and (R)-1,1-Dimethylethyl[[5-methoxy-2-[3-oxo-3-[2-oxo-4-(phenylmethyl)-3-oxazolidinyl]propyl]phenyl]methyl](2-phenylethyl)carbamate respectively. This mixture was used without further purification.

e) Methyl (S)-8-methoxy-3-oxo-2-(2-phenylethyl)-2,3,4,5-tetrahydro-1H-2-benzazepine-4-acetate

To a solution of [R-(R*, S*)]-methyl β-[[4-methoxy-2-[[[(1,1-dimethylethoxy)carbonyl](2-phenylethyl)amino]methyl]phenyl]methyl]-γ-oxo-4-(phenylmethyl)-3-oxazolidinebutanoate (9.91 g, 15.4 mmole) in THF (75 mL) was added a solution of lithium hydroxide monohydrate (646 mg, 15.4 mmole) and H₂O₂ (5.2 ml, 30% in H₂O, 46.2 mmole) in H₂O (25 mL) at 0° C. over 10 minutes. After 1.5 hr a solution of Na₂SO₃ (9.7 g, 77 mmole) in H₂O (100 mL) was added. The mixture was acidified to pH 4 using 2 M HCl and extracted with EtOAc (3×200 mL). The combined EtOAc layers were dried over MgSO₄ and concentrated. The resulting residue was dissolved in 4.0 M HCl in dioxane (75 mL). After 45 minutes the mixture was concentrated then reconcentrated from toluene (200 mL).

The above residue was dissolved in anhydrous DMF (75 mL). To this solution was added NaHCO₃ (6.50 g 77.0 mmole) and triethylamine (4.3 mL, 30.8 mmole) at RT. The mixture was cooled to 0° C. and diphenylphosphoryl azide (5 mL, 23.1 mmole) was added. After 16 hr the mixture was concentrated. The resulting paste was dissolved in EtOAc-(500 mL), washed with H₂O (2×300 mL), dried over MgSO₄, and concentrated. Flash silica gel chromatography (40% EtOAc/hexanes) gave the title compound (2.61 g).

f) Methyl (S)-8-hydroxy-3-oxo-2-(2-phenylethyl)-2,3,4,5-tetrahydro-1H-2-benzazenine-acetate

To a solution of methyl (S)-8-methoxy-3-oxo-2-(2-phenylethyl)-2,3,4,5-tetrahydro-1H-2-benzazepine4-acetate (2.61 g, 7.1 mmole) in CH₂Cl₂ (40 mL) was added BBr₃ (21.3 mL, 1M in CH₂Cl₂, 21.3 mmole) at −20° C. After 45 minutes the mixture was quenched with MeOH (200 mL) and concentrated. The residue was filtered through a silica gel plug using 50% EtOAc/hexanes as eluent. The resulting orange solid was recrystallized from MeOH/H₂O to give the title compound as an off-white solid (2.16 g 81I%).

### Preparation 8

Preparation of 2-[(3-hydroxy-1-propyl)amino]pyridine-N-oxide

a) 2-[(3-Hydroxy-1-propyl)amino]pyridine-N-oxide

A mixture of 2-chloropyridine-N-oxide (16.6 g, 0.1 mole), 3-amino-1-propanol (15.3 mL, 0.2 mole), NaHCO₃ (42 g, 0.5 mole), and tert-amyl alcohol (100 mL) was heated to reflux. After 21 hr, the reaction was cooled, diluted with CH₂,Cl₂ (300 mL), and suction filtered to remove insoluble materials. The filtrate was concentrated and reconcentrated from toluene to leave a yellow oil. Silica gel chromatography ( 20% MeOH/CHCl₃) gave the title compound (15.62 g, 93%) as a yellow solid.

### Preparation 9

Preparation of 3-(4-nitrobenzyloxycarbonylamino)-1-propanol

a) 3-(4-Nitrobenzyloxycarbonylamino)-1-propanol

To a solution stirred under argon at room temperature of 3-amino-1-propanol (0.77 g, 1.1 mmol) and triethylamine (2.85 mL, 7 mmol) in THF (5 mL) was added a suspension of 4nitrobenzyl chloroformate (2 g, 1 mmol) in THF (20 mL). The resulting mixture was allowed to stir at room temperature over the weekend, then was concentrated. The residue was purified by chromatography on silica gel (0%-2% MeOH/CH₂Cl₂) to give the title compound (0.80 g, 34%) as a pale yellow oil:

### Preparation 10

Preparation of 6-(methylamino)-2-pyridylethanol

a) 2-(tert-Butoxycarbonylamino)-6-picoline

To a stirred solution of 2-amino-6-picoline (4.33 g, 40 mmol), Et₃N (6.2 mL, 40 mmol) and CH₂Cl₂ (50 mL) at 0° C. was added di-tert-butyl dicarbonate (9.6 g, 44 mmol). After stirring at RT overnight, the reaction mixture was concentrated in vacuum, diluted with H₂O and extracted with CH₂Cl₂ (2×50 mL). Drying (MgSO₄) and concentration gave the title compound as a colorless oil.

b) 2-[(tert-Butoxycarbonyl)methylamino]-6-picoline

To the suspension of NaH (60% dispersion in mineral oil, 0.44 g, 11 mmol) in DMF (20 mL) at 0° C. was added a solution of 2-(tert-butoxycarbonylamino)-6-picoline (2.1 g,10 mmol) in DMF (30 mL). The reaction was stirred at 0° C. for 15 min; then methyl iodide (1.6 g, 11 mmol) was added. The reaction mixture was concentrated in vacuum, diluted with H₂O, and extracted with CH₂Cl₂ (3×50 mL). Drying (MgSO₄) and concentration gave the title compound as a colorless oil.

c) Ethyl-6-[(tert-butoxycarbonyl)methylamino]-2-pyridylacetate

LDA (18 mmol) was prepared in THF (30 mL), cooled to −78° C., and 2-[(tert-butoxycarbonyl)methylamino]-6-picoline (2 g, 9 mmol) was added, forming a deep red solution. After 15 min, diethylcarbonate (18 mL, 15 mmol) was added. The burgundy-colored solution was stirred at −78° C. for an additional 15 min, then the reaction was quenched with saturated NH₄Cl solution. The mixture was warmed to RT and extracted with EtOAc (3×30 mL). The combined organic layers were washed with brine, dried (MgSO₄), filtered and concentrated. Silica gel chromatography gave the title compound as a colorless oil.

d) Ethyl-6-(methylamino)-2-pyridylacetate

A solution of ethyl-6-[(tert-butoxycarbonyl)methylamino]-2-pyridylacetate (0.6 g, mmol) and 4 M HCl/dioxane (5 mL, 20 mmol) was stirred at RT overnight, then was concentrated. Reconcentration from toluene gave the title compound as white solid.

e) 6-(Methylamino)-2-pyridylethanol

To a mechanically stirred solution of LiAlH₄ in THF (1.0 M, 20 mL, 20.4 mmol) was added dropwise a solution of ethyl-2-(methylamino)-6-pyridylacetate (0.38 g,2 mmol) in THF (10 mL). After the addition was completed, the reaction mixture was warmed to 0° C. and quenched with 10% NaOH solution. The solids were removed by filtration, and the filtrate was concentrated in vacuum. The residue was dissolved in CH₂Cl₂ and the solution was dried (MgSO₄) and concentrated. Reconcentration from toluene (3 x) gave the title compound as a colorless oil.

### Preparation 11

Preparation of ethyl(±)-10,11-dihydro-3-hydroxy-5H-dibenzo[a,d]cycloheptene-10-acetate

a) 6Methoxy-1-phenylindene

A solution of 3.0 M phenylmagnesium bromide in Et₂O (680 mL, 2.04 mole) under argon at ambient temperature was diluted with Et₂O (700 mL) with stirring, and a solution of 6-methoxy-1-indanone (277 g, 1.71 mole) in THF (1400 mL) was added dropwise over 1 hr. The reaction mixture was stirred for 2 h at ambient temperature and then was poured with stirring into saturated NH₄Cl (2.8 L). H₂O (1.4 L) was added, and the organic phase separated. The aqueous phase was extracted with Et₂O (2 x I L), and the combined organic extracts were concentrated to give crude 6-methoxy-1-phenyl-1-indanol (445 g) as a brown oil. This oil was dissolved in toluene (2.5 L), and p-toluenesulfonic acid monohydrate (12.3 g, 0.065 mole) was added. The solution was stirred and heated at reflux for 16 hr using a Dean-Stark trap with a condenser. H₂O collection was minimal after 2 h and totaled 28 mL. The solution was cooled and extracted sequentially with 5% aqueous Na₂CO₃ (1 L) and H₂O (2×1 L). The organic layer was concentrated to give a dark brown oil (400 g). This oil was distilled under vacuum to give the title compound (298.2 g, 79%) as a yellow oil.

b) 2-Benzoyl-4-methoxyphens,acetic acid

Acetone (4.2 L) was chilled to 10° C., and a solution of 6-methoxy-1-phenylindene (271 g, 1.22 mole) in acetone (1.8 L) was added over 1.5 hr concurrently with Jones reagent (1.8 L, prepared from CrO₃ (470 g, 4.70 mole), H₂O (1 L), and conc H₂SO₄ (405 mL)). 4% Aqueous OsO₄ (153 mL) was added to the resulting mixture in two portions, one at the onset of addition and the second at the mid-point of the addition, maintaining the temperature of the reaction mixture below 15° C. Following the addition, the reaction mixture was warmed to 22° C. and stirred for 1.5 h, during which time a mild exotherm increased the temperature to 28° C. The reaction mixture was then cooled to below 20° C. and isopropanol (1 L) was added, dropwise initially and rapidly after the initial exotherm diminished. Stirring became difficult during this phase. The temperature reached 32° C. during the isopropanol addition. H₂O (2 L) was added and the mixture was transferred to a separatory funnel. Additional H₂O was added to dissolve the precipitated chromous acid, and the mixture was extracted with CH₂Cl₂ (2 L). The organic (upper) layer was separated and the aqueous phase was extracted with CH₂Cl₂ (2×1 L). The combined CH₂Cl₂ extracts were washed sequentially with H₂O (2 L) and saturated brine (2 L), and then were concentrated to give a moist gray solid (416 g). This was triturated with a mixture of acetone and EtOAc and filtered and dried to give the title compound (225.4 g, 71%) as an off-white solid.

c) 2-Benzyl-4-methoxyphenylacetic acid

2-Benzoyl4-methoxyphenylacetic acid (215.5 g, 0.80 mole) was divided into two equal portions, and each was dissolved in glacial AcOH (1.5 L) in a 2.5 L pressure bottle. 5% Pd/C (10 g, 0.0048 mole) was added to each, and each mixture was shaken at ambient temperature under hydrogen on a Parr apparatus. After 2.5 hr, the mixtures were filtered to remove the catalyst, and the filter pads were washed with EtOAc. The combined filtrates were concentrated to give the title compound (215 g, quantitative) as a heavy yellow oil which crystallized on standing.

d) 10,11-Dihydro-3-methoxy-5H-dibenzo[a,d]cyclohepten-10-one

A solution of 2-benzyl-4-methoxyphenylacetic acid (215 g of crude material that contained 204.6 g (0.80 mole) of pure material) in CH₂Cl₂ (1 L) was stirred under argon at ambient temperature, and DMF (1 mL) was added, followed by oxalyl chloride (400 mL, 4.59 mole). The oxalyl chloride was added over 1 hr, dropwise initially to control the vigorous gas evolution. The solution was stirred for 16 h at ambient temperature and then was concentrated to give the crude acid chloride (207.7 g, 0.756 mol, 95%) as a yellow liquid. This liquid was dissolved in CH₂Cl₂ to a total volume of 500 mL, and the solution and AlCl₃ (100.8 g, 0.756 mol) were added concurrently over 1 hr to CH₂Cl₂ (3.7 L) with stirring under argon at ambient temperature. The temperature was 28° C. at the completion of the addition. The reaction mixture was stirred for 16 h at ambient temperature, during which time a solid precipitated. H₂O (1 L) was added, initially dropwise, over a period of 30 min. The mixture was then separated and the organic phase was washed sequentially with H₂O (1 L) and 5% aqueous NaHCO₃ (1 L). The CH₂Cl₂ solution was then concentrated to give a yellow solid (175.3 g). Recrystallization from EtOAc/hexane gave the title compound (128 g, 71%).

e) Ethyl (±)-10,11-dihydro-10-hydroxy-3-methoxy-5H-dibenzo[a,d]cycloheptene-10-acetate

A 1.0 M solution of lithium bis(trimethylsilyl)amide in hexanes (1282 mL, 1.282 mole) was added to THF (4.0 L) at −70° C. under argon, then EtOAc (146 mL, 1.49 mole) was added dropwise over 20 min. The reaction mixture was allowed to stir for 15 min, then N,N,N′,N′-tetramethylethylenediamine (378 mL, 2.5 mole) was added over 20 min. The reaction mixture was stirred for 10 min, then a solution of 10,11-dihydro-3-methoxy-5H-dibenzo[a,d]cyclohepten-10-one (119.2 g, 0.50 mol) in anhydrous THF (1.26 L) was added dropwise over 40 min. The temperature was maintained below −65° C. during all of these additions. The reaction mixture was stirred for 20 min at −65 to −70° C. and then was poured into saturated aqueous NH₄Cl (6.2 L) with vigorous stirring. The organic layer was separated and the aqueous phase was extracted with EtOAc (2×1 L). The combined organic extracts were washed with H₂O (2×1 L) and then were concentrated to give a light brown oil (175 g). Thin-layer chromatography (20% EtOAc/hexanes) showed R_{f} 0.5 major (desired product) and R_{f} 0.7 minor (recovered ketone). The crude product was chromatographed on silica gel (2 kg, 10% EtOAc/hexanes) to afford the title compound (101 g, 61%) as a yellow oil.

f) Ethyl (±)-10,11-dihydro-3-methoxy-5H-dibenzo[a,d]cycloheptene-10-acetate

Ethyl (±)-10,11-dihydro-10-hydroxy-3-methoxy-5H-dibenzo[a,d]cycloheptene-10-acetate (101 g, 0.31 mole) was dissolved in glacial acetic acid (1.8 L) and 12 N HCl (28.5 mL, 0.34 mole) was added. The mixture was placed in a 2.5 L pressure bottle containing 5% Pd/C (20 g, 0.0094 mole), and the resulting mixture was shaken at 35° C. under hydrogen on a Parr hydrogenation apparatus equipped with a jacket heater. After 18 hr. the reaction was cooled to ambient temperature, and the catalyst was removed by filtration. The filtrate was concentrated to give a light yellow oil (85.1 g). This was chromatographed on silica gel (2 kg, step-gradient with 5% to 10% EtOAc/hexanes) to afford the title compound (69.1 g, 72%) as an oil.

g) Ethyl (±)-10,11-dihydro-3-hydroxy-5H-dibenzo[a,d]cycloheptene-10-acetate

A solution of ethyl (+)-10,11-dihydro-3-methoxy-5H-dibenzo[a,d]cycloheptene-10-acetate (8.5 g, 0.027 mole) in CH₂Cl₂ (150 mL) was chilled to −10° C. with stirring under argon. Ethanethiol (10.7 mL, 0.144 mole) was added, followed by AlCl₃ (20.6 g, 0.154 mole) in two portions over 15 min. An exotherm increased the temperature to 0° C. following the additions, and the temperature was then increased to 25° C. using a water bath. The reaction mixture was stirred at 25 to 30° C. for 2.25 hr, at which point it was poured into ice-H₂O. The organic layer was separated, methanol (100 mL) was added, and the mixture was extracted with CH₂Cl₂ (2×50 mL). The combined CH₂Cl₂ extracts were washed with H₂O (250 mL) and then were concentrated to give a viscous oil (8.6 g). This was taken up in Et₂O (150 mL) and the ether was boiled off while replacing it with hexane. The desired phenol first separated as an oil which crystallized on stirring at ambient temperature. Two crops of solid were collected to afford the title compound (7.1 g, 89%).

### Preparation 12

HPLC separation of the enantiomers of ethyl (±)-10,11-dihydro-3-hydroxy-5H-dibenzo[a,d]cycloheptene-10-acetate

a) Ethyl (R)-(+)-10,11-dihydro-3-hydroxy-5H-dibenzo[a,d]cycloheptene-10-acetate and ethyl (S)-(-)-10,11-dihydro-3-hydroxy-5H-dibenzo[a,d]cycloheptene-10-acetate

Ethyl (±)-10,11-dihydro-3-hydroxy-5H-dibenzo[a,d]cycloheptene-10-acetate was resolved into its enantiomers using the following conditions: Daicel Chiralcel OJ® column (21.2×250 mm), 20% ethanol in hexane mobile phase, 15 mL/min flow rate, uv detection at 254 nm, 140 mg injection; t_{R} for ethyl (S)-(-)-10,11-dihydro-3-hydroxy-5H-dibenzo[a,d]cyclohexene-10-acetate=10.4 min.; t_{R} for ethyl (R)-(+)-10,11-dihydro-3-hydroxy-5H-dibenzo[a,d]cyclohexene-10-acetate=13.1 min.

### EXAMPLE 1

Preparation of (±)-7-[[[(2-benzimidazolyl)methyl]amino]carbonyl]-4-methyl-3-oxo-2,3,4,5-tetrahydro-1H-1,4-benzodiazepine-2-acetic acid

a) Methyl (±)-7-[[[(2-benzimidazolyl)methyl]amino]carbonyl]-4-methyl-3-oxo-2,3,4,5 -tetrahydro-1H-1,4-benzodiazepine-2-acetate

A mixture of methyl (±)-7-carboxy-4-methyl-3-oxo-2,3,4,5-tetrahydro-1H-1,4-benzodiazepine-2-acetate (0.57 g, 1.82 mmol) and thionyl chloride (15 mL) was refluxed for 1 h. The resulting orange solution was concentrated to dryness to leave a yellow-orange foam. This was dissolved in CH₂Cl₂ (10 mL) and added dropwise to a solution containing 2-(aminomethyl)benzimidazole dihydrochloride (1.2 g, 5.46 mmol), pyridine (0.72 g, 9.1 mmol), and triethylamine (0.55 g, 5.46 mmol) in CH₂Cl₂ (15 mL) at 0° C. under argon. The reaction mixture was then stirred in RT under argon. After 25.5 h, CH₂Cl₂ (200 mL) and 5% NaHCO₃ (50 mL) were added to the reaction mixture to give a light yellow precipitate which was filtered and air-dried to give the title compound (0.11 g, 14%). The filtrate was separated and the organic layer was washed sequentially with 5% NaHCO₃ (50 mL) and H₂O (50 mL), then was concentrated on the rotavap. After trituration with CH₂Cl₂ and air-drying, a yellowish solid was collected to yield more of the title compound (0.35 g, 45%).

b) (±)-7-[[[(2-Benzimidazolyl)methyl]amino]carbonyl]-4-methyl-3-oxo-2,3,4,5-tetrahydro-1H-1,4-benzodiazepine-2-acetic acid

1.0 N LiOH (0.57 mL, 0.57 mmol) was added dropwise at RT to a mixture of methyl (±)-7-[[[(2-benzimidazolyl)methyl]amino]carbonyl]-4-methyl-3-oxo-2,3,4,5-tetrahydro-1H-1,4-benzodiazepine-2-acetate (0.11 g, 0.26 mmol) in THF (4 mL) and H₂O (5 mL). The resulting light brownish-yellow solution was stirred for 21.5 h, then was concentrated on the rotavap. The resulting residue was lyophilized to give the crude product (0.11 g, 100%) as a yellowish powder. Preparative HPLC (PRP-1® column, step gradient, 10-20% CH₃CN/H₂O-0.1% TFA) afforded the title compound.

### EXAMPLE 2

Preparation of (±)-7-[[[(2-benzimidazoyl)methyl]amino]carbonyl]-3-oxo-4-(2-phenylethyl)-2,3,4,5-tetrahydro-1H-1,4-benzodiazepine-2-acetic acid

a) Methyl (±)-7-[[[(2-benzimidazolyl)methyl]amino]carbonyl]-3-oxo-4-(2-phenylethyl)-2,3,4,5-tetrahydro-1H-1,4-benzodiazepine-2-acetate

EDC (230 mg, 1.2 mmol) was added to a stirred solution of methyl (±)-7-carboxy-3-oxo-4-(2-phenylethyl)-2,3,4,5-tetrahydro-1H-1,4-benzodiazepine-2-acetate (382.4 mg. 1.0 mmol), 2-(aminomethyl)benzimidazole dihydrochloride (264 mg, 1.2 mmol), HOBT.H₂O (162 mg, 1.2 mmol), and diisopropylethylamine (0.70 mL, 4.0 mmol) in anhydrous DMF (5 mL) at RT. After 19 h, the reaction was concentrated on the rotavap (high vacuum), and the residue was partitioned between H₂O (5 mL) and EtOAc (20 mL). The layers were separated and the organic layer was washed with H₂O (5 mL). Drying (MgSO₄), concentration, and silica gel chromatography (load with 5% MeOH/CHCl₃; gradient: 5% MeOH in 1:1 EtOAc/CHCl₃ (300 mL), then 10% MeOH/EtOAc (400 mL), then 10% MeOH/CHCl₃) gave the title compound (414.9 mg, 81%) as an off-white solid.

b) (±)-7-[[[(2-Benzimidazolyl)methyl]amino]carbonyl]-3-oxo4-(2-phenylethyl)-2,3,4,5-tetrahydro-1H-1,4-benzodiazepine-2-acetic acid

A mixture of methyl (±)-7[[[(2-benzimidazolyl)methyl]amino]carbonyl-3-oxo-4-(2-phenylethyl)-2,3,4,5-tetrahydro-1H-1,4-benzodiazepine-2-acetate (413.1 mg, 0.81 mmol), 1.0 N LiOH (0.97 mL, 0.97 mmol), THF (4 mL), and H₂O (3 mL) was stirred at 40-45° C. for 20 min, and the resulting solution was stirred at RT for 17 h. Acidification with TFA (0.19 mL, 2.4 mmol) and concentration left an off-white solid. Recrystallization from CH₃CN/H₂O gave the title compound (343.2 mg, 69%) as a colorless powder.

### EXAMPLE 3

Preparation of (±)-4-isopropyl-7-[[[(2-benzimidazolyl)methyl]amino]carbonyl]-3-oxo-2,3,4,5-tetrahydro-1H-1,4-benzodiazepine-2-acetic acid

a) Methyl (±)-4-isopropyl-7-[[[(2-benzimidazolyl)methyl]amino]carbonyl]-3-oxo-2,3,4,5-tetrahydro-1H-1,4-benzodiazepine-2-acetate

EDC (173 mg, 0.90 mmol) was added to a stirred solution of methyl (±)-7-carboxy-4-isopropyl-3-oxo-2,3,4,5-tetrahydro-1H-1,4-benzodiazepine-2-acetate (240.3 mg, 0.75 mmol), 2-(aminomethyl)benzimidazole dihydrochloride (198 mg, 0.90 mmol), HOBT.H₂O (122 mg, 0.90 mmol), and diisopropylethylamine (0.52 mL, 3.0 mmol) in anhydrous DMF (4 mL) at RT. After 20 h, the reaction was concentrated on the rotavap (high vacuum), and the residue was diluted with H₂O (5 mL) to afford a gummy precipitate. EtOAc (3 mL) was added and the mixture was stirred briskly. The precipitate remained gummy, but changed in form so that it was suspended as a mass in the solvents. The solvents were drawn off with a pipet and the residue was suspended in MeOH (3 mL) and EtOAc (6 mL). The mixture was stirred briskly at RT for several min, then was cooled in ice and filtered. The filter pad was washed with EtOAc and dried in high vacuum to leave the title compound (275.1 mg, 82%) as an off-white powder.

b) (±)-4-Isopropyl-7-[[[(2-benzimidazolyl)methyl]amino]carbonyl]-3-oxo-2,3,4,5-tetrahydro-1H-1,4-benzodiazepine-2-acetic acid

A mixture of methyl (±)4-isopropyl-7-[[[(2-benzimidazolyl)methyl]amino]carbonyl]-3-oxo-2,3,4,5-tetrahydro-1H-1,4-benzodiazepine-2-acetate (275.1 mg. 0.61 mmol), 1.0 N LiOH (0.73 mL, 0.73 mmol), THF (3 mL), and H₂O (2.3 mL) was stirred at 35° C. for 45 min, and the resulting solution was stirred at RT. After 17.5 h, the solution was filtered, and the filtrate was neutralized with 1.0 N HCl (0.73 mL). Since the product did not precipitate, the solution was acidified with TFA (0.2 mL) and concentrated. The resulting solid was triturated with H₂O to leave a nearly colorless solid, which was dissolved with warming in 1:1 CH₃CN/H₂O. The solution was cooled to RT and diluted with several volumes of H₂O/0.1% TFA. ODS chromatography (20% CH₃CN/H₂O-0.1% TFA), concentration, and lyophilization gave the title compound (293.4 mg, 80%) as a colorless powder.

### EXAMPLE 4

Preparation of (2S)-7-[[[(2-Benzimidazolyl)methyl]amino]carbonyl]-4-methyl-3-oxo-2,3,4,5-tetrahydro-1H-1,4-benzodiazepine-2-acetic acid

a) Methyl (2S)-7-[[[(2-benzimidazolyl)methyl]amino]carbonyl]-4-methyl-3-oxo-2,3,4,5-tetrahydro-1H-1,4-benzodiazepine-2-acetate

EDC (1.15 g, 6.02 mmol) was added to a solution of methyl (2S)-7-carboxy-4-methyl-3-oxo-2,3,4,5-tetrahydro-1H-1,4-benzodiazepine-2-acetate (2.11 g, 5.02 mmol), 2-aminomethylbenzimidazole dihydrochloride (1.15 g, 6.02 mmol), HOBT.H₂O (811 mg, 6.02 mmol), and diisopropylethylamine (1.76 mL, 10 mmol) in anhydrous DMF (25 mL) at RT. After 21 h, the reaction was concentrated on the rotavap (high vacuum), and the residue was taken up in CH₂Cl₂ (240 mL) and washed with H₂O. The organic layer was dried (Na₂SO₄), dissolved in xylenes, and reconcentrated to remove residual DMF. The crude product was chromatographed on silica gel (MeOH/CHCl₃) to give the title compound (1.1 g, 52%).

b) (2S)-7-[[[(2-Benzimidazolyl)methyl]amino]carbonyl]-4-methyl-3-oxo-2,3,4,5-tetrahydro-1H-1,4-benzodiazepine-2-acetic acid

1 N NaOH (4.75 mL, 4,75 mmol) was added to a cold solution of methyl (2S)-7-[[[(2-benzimidazolyl) methyl]amino]carbonyl]-4-methyl-3-oxo-2,3,4,5-tetrahydro-1H-1,4-benzodiazepine-2-acetate (1.0 g, 2.38 mmol), MeOH (10 mL) and H₂O (5 mL). The solution was stirred at room temperature for 18 hr and concentrated. ODS chromatography (CH₃CN/H₂O-0.1% TFA) gave the title compound (0.91 g 94%).

### EXAMPLE 5

Preparation of (±)-8-[[[(2-Benzimidazolyl)methyl]amino]carbonyl]-2-methyl-3-oxo-2,3,4,5-tetrahydro-1H-2-benzazepine-4-acetic acid

a) Methyl (±)-8-[[[(2-benzimidazolyl)methyl]amino]carbonyl]-2-methyl-3-oxo-2,3,4,5-tetrahydro-1H-2-benzazepine-4-acetate

To a solution stirred under argon at room temperature of methyl (±)-8-carboxy-2-methyl-3-oxo-2,3,4,5-tetrahydro-1H-2-benzazepine-4-acetate (0.30 g, 1 mmol). 2-aminomethylbenzimidazole dihydrochloride (0.27 g, 1.2 mmol), HOBT.H₂O (0.17 g, 1.2 mmol), diisopropylethylamine (0.53 g, 4 mmol), and DMF (5 mL) was added EDC (0.24 g, 1.2 mmol). The resulting mixture was stirred for 18 h, then was concentrated to dryness, and the residue was partitioned between EtOAc and H₂O. The organic phase was washed twice with H₂O and once with brine, dried (MgSO₄), and concentrated. The residue was recrystallized from boiling EtOAc to give the title compound (0.16 g. 37%) as a colorless solid.

b) (±)-8-[[[(2-Benzimidazolyl)methyl]amino]carbonyl]-2-methyl-3-oxo-2,3,4,5-tetrahydro-1H-2-benzazepine4-acetic acid

A solution of methyl (±)-8-[[[(2-benzimidazolyl)methyl]amino]carbonyl]-2-methyl-3-oxo-2,3,4,5-tetrahydro-1H-2-benzazepine4-acetate (0.10 g, 0.24 mmol). LiOH H₂O (0.013 g, 0.31 mmol), THF (2 mL), and H₂O (2 mL) was stirred at RT for 18 h, then was concentrated to dryness. The residue was dissolved in H₂O, and the solution was brought to pH 4-5 with 3N HCl. The resulting precipitate was collected by filtration and dried. Recrystallization from boiling isopropanol gave the title compound (0.035 g, 36%) as a colorless solid.

### EXAMPLE 6

Preparation of (±)-8-[[[N-benzimidazolyl)methyl-N-methyl]amino]carbonyl]-2-methyl-3-oxo-2,3,4,5-tetrahydro-1H-2-benzazepine-4-acetic acid

a) Methyl (±)-8-[[[N-(2-benzimidazolyl)methyl-N-methyl]amino]carbonyl]-2-methyl-3-oxo-2,3,4,5-tetrahydro-1H-2-benzazepine-4-acetate

Following the procedure of Example 5(a), methyl (±)-8-carboxy-2-methyl-3-oxo-2,3,4,5-tetrahydro-1H-2-benzazepine-4acetate was coupled with 2-(methylamino)methylbenzimidazole. Chromatography on silica gel (5% MeOH/CH₂Cl₂) gave the title compound (67%) as a colorless foam.

b) (±)-8-[[[N-(2-Benzimidazolyl)methyl-N-methyl]amino]carbonyl]-2-methyl-3-oxo-2,3,4,5-tetrahydro-1H-2-benzazepine4-acetic acid

Following the procedure of Example 5(b), methyl (±)-8-[[[N-(2-benzimidazolyl)methyl-N-methyl]amino]carbonyl]-2-methyl-3-oxo-2,3,4,5-tetrahydro-1H-2-benzazepine-4-acetate was saponified. Extraction with CH₂Cl₂, concentration, and drying gave the title compound (52%) as a colorless solid.

### EXAMPLE 7

Preparation of (±)-7-[[[N-(2-benzimidazolyl)methyl-N-methyl]amino]carbonyl]-3-oxo-4-(2-phenylethyl)-2,3,4,5-tetrahydro-1H-1,4-benzodiazepine-2-acetic acid

a) Methyl (±)-7-[[[N-(2-benzimidazolyl)methyl-N-methyl]amino]carbonyl]-3-oxo-4-(2-phenylethyl)-2,3,4,5-tetrahydro-1H-1,4-benzodiazepine-2-acetate

Following the procedure of Example 5(a), methyl (±)-7-carboxy-3-oxo4-(2-phenylethyl)-2,3,4,5-tetrahydro-1H-1,4-benzodiazepine acetate and 2-(methylamino)methylbenzimidazole were coupled. Chromatography on silica gel (1%-5% MeOH/CH₂Cl₂) gave the title compound (57%) as a colorless solid.

b) (±)-7- [[[N-(2-Benzimidazolyl)methyl-N-methyl]amino]carbonyl]-3-oxo-4-(2-phenylethyl)-2,3,4,5-tetrahydro-1H-1,4-benzodiazepine-2-acetic acid

Methyl (±)-7-[[[N-(2-benzimidazolyl)methyl-N-methyl]amino]carbonyl]-3-oxo-4-(2-phenylethyl)-2,3,4,5-tetrahydro-1H-1,4-benzodiazepine-2-acetate was saponified according to the procedure of Example 5(b). Recrystallization from boiling isopropanol gave the title compound (57%) as a colorless solid.

### EXAMPLE 8

Preparation of (±)-7-[[[N-(2-benzimidazolyl)methyl-N-methyl]amino]carbonyl]-4-methyl-3-oxo-2,3,4,5-tetrahydro-1H-1,4-benzodiazepine-2-acetic acid

a) 2-(Methylaminomethyl)benzimidazole dihydrochloride

Methylamine (5.0 g, 0.16 mole) was dissolved in a solution of Et₂O (100 mL) and EtOH (5 mL) at 0° C., and 2-chloromethylbenzimidazole (13.4 g, 0.08 mole) was added in small portions. The reaction mixture was stirred at RT for 3 h, then was allowed to stand at RT overnight. More Et₂O (200 mL) was added, and the reaction was cooled in an ice bath for 3 h before filtering off the precipitate. The filtrate was saturated with HCl and filtered, and the filtrate was concentrated. Silica gel chromatography (step gradient, 10-25% MeOH/CH₂Cl₂) yielded the title compound (2.5 g, 13%).

b) Methyl (±)-7-[[[N-(2-benzimidazolyl)methyl-N-methyl]amino]carbonyl]-4-methyl-3-oxo-2,3,4,5-tetrahydro-1H-1,4-benzodiazepine-2-acetate

Following the procedure of Example 1(a), except substituting 2-(methylamino methyl)benzimidazole dihydrochloride (1.2 , 5.13 mmol) for the 2-(aminomethyl) benzimidazole dihydrochloride, the crude title compound was prepared. Silica gel chromatography (10% MeOH/CH₂Cl₂) yielded the title compound (0.29 g, 39%) as an off-white solid.

c ) (±)-7-[[[N -(2-Benzimidazolyl)methyl-N-methyl]amino]carbonyl]-4-methyl-3-oxo-2,3,4,5-tetrahydro-1H-1,4-benzodiazepine-2-acetic acid

Following the procedure of Example 1(b), the compound of Example 8(b) was saponified and purified to give the title compound (0.21 g, 80%).

### EXAMPLE 9

Preparation of (2S)-7-[[[N-(2-benzimidazolyl)methyl-N-methyl]amino]carbonyl]-4-methyl-3-oxo-2,3,4,5-tetrahydro-1H-1,4-benzodiazepine-2-acetic acid

a) Methyl (2S)-7-[[[N-(2-benzimidazolyl)methyl-N-methyl]amino]carbonyl]-4-methyl-3-oxo-2,3,4,5-tetrahydro-1H-1,4-benzodiazepine-2-acetate

Diisopropylethylamine (0.29 g, 2.25 mmol) was added in one portion to a stirred mixture of 2-(methylaminomethyl)benzimidazole bis(trifluoroacetate) (1.8 mmol), methyl (2S)-7-carboxy4-methyl-3-oxo-2,3,4,5-tetrahydro-1H-1,4-benzodiazepine-2-acetate (0.44 g, 1.50 mmol), EDC (0.34 g, 1.8 mmol) and HOBT.H₂O (0.24 g. 1.8 mmol) in DMF (8 mL) at RT under argon. After 24 h. the solution was poured into a mixture of ice-water (90 g) and 5% NaHCO₃ (10 mL). The resulting precipitate was filtered and air-dried. Flash chromatography (silica gel, MeOH/CH₂Cl₂) yielded the title compound (79%).

b) (2S)-7-[[[N-(2-Benzimidazolyl)methyl-N-methyl]amino]carbonyl]-4-methyl-3-oxo-2,3,4,5-tetrahydro-1H-1,4-benzodiazepine-2-acetic acid

Following the procedure of Example 1(b), the compound of Example 9(a) was saponified and purified to give the title compound (0.11 g, 91%).

### EXAMPLE 10

Preparation of (±)-4-Methyl-7-[[[N-(2-(1-methyl)benzimidazolyl)methyl-N-methyl]amino]carbonyl]-3-oxo-2,3,4,5-tetrahydro-1H-1,4-benzodiazepine-2-acetic acid

a) 2-[[N-(tert-Butoxycarbonyl)-N-methyl]aminomethyl]benzimidazole

Di-tert-butyl dicarbonate (1.12 g, 5.13 mmol) was added dropwise at 0° C. to a mixture containing 2-(methylaminomethyl)benzimidazole dihydrochloride (1.0 g, 4.27 mmol), dioxane (25 mL), H₂O (25 mL), and 1 N NaOH (12.8 mL, 12.8 mmol). After 2 h, the reaction was warmed to RT and stirred for 21 h. The solvent was evaporated on the rotavap, and the pH was adjusted to 5 using 1 M NaHSO₄. The mixture was extracted with CH₂Cl₂ (2×80 mL), and the combined organic layers were washed with brine (30 mL) and dried (MgSO₄). Concentration gave the title product (0.7 g, 64%).

b) 1-Methyl-2-[[N-(tert-butoxycarbonyl)-N-methyl]aminomethyl]benzimidazole

A mixture of 2-[[N-(tert-butoxycarbonyl)-N-methyl]aminomethyl]benzimidazole (0.51 g, 1.95 mmol), NaH (0.12 g, 5.0 mmol), DMF (5 mL), and THF (20 mL) was stirred at RT under argon for 5 min, then methyl iodide (0.83 g, 5.86 mmol) was added. The reaction mixture was stirred at RT for 170 min, then was concentrated on the rotavap. The residue was diluted with CH₂Cl₂ (100 mL), and the mixture was washed sequentially with H₂O (30 mL), 5% NaHCO₃ (30 mL), and brine (30 mL). Drying (Na₂SO₄) and concentration gave the title compound (0.51, 94%).

c) 1-Methyl-2-(methylaminomethyl)benzimidazole bis(trifluoroacetate)

A mixture of 1-methyl-2-[[N-(tert-butoxycarbonyl)-N-methyl]aminomethyl]benzimidazole (0.51 g, 1.85 mmol) in 25% TFA/CH₂Cl₂ (20 mL) was stirred at RT under argon for 20 min. The solvent was removed on the rotavap and the residue was recrystallized from Et₂O/CH₂Cl₂ to give title compound (0.69 g, 92%).

d) Methyl (±)-4-methyl-7-[[[N-(2-(1-methyl)benzimidazolyl)methyl-N-methyl]amino]carbonyl]-3-oxo-2,3,4,5-tetrahydro-1H-1,4-benzodiazepine-2-acetate

Following the procedure of Example 9(a), except substituting 1-Methyl-2-(methylaminomethyl)benzimidazole bis(trifluoroacetate) for 2,6-diaminopyridine, the title compound (0.53 g, 77%) was prepared.

e) (±)-4-Methyl-7-[[[N-(2-(1-methyl)benzimidazoyl)methyl-N-methyl]amino]carbonyl]-3-oxo-2,3,4,5-tetrahydro-1H-1,4-benzodiazepine-2-acetic acid

Following the procedure of Example 1(b), the compound of Example 10(d) was saponified and purified to give the title compound (0.13 g, 60%).

### EXAMPLE 11

Preparation of (±)-7-[[[2-(benzimidazolyl)methyl]methyl]amino]carbonyl]-4-(2-methoxyethyl)-3-oxo-2,3,4,5-tetrahydro-1H-1,4-benzodiazepine-2-acetic acid

a) Methyl (±)-7-[[[2-(benzimidazolyl)methyl]methylamino]carbonyl]-4-(2-methoxyethyl)-3-oxo-2,3,4,5-tetrahydro-1H-1,4-benzodiazepine-2-acetate

EDC (138 mg, 0.72 mmol) was added to a solution of methyl (±)-7-carboxy-4-(2-methoxyethyl)-3-oxo-2,3,4,5-tetrahydro-1H-1,4-benzodiazepine-2-acetate (202 mg, 0.6 mmol), 2-(methylaminomethyl)benzimidazole dihydrochloride (0.72 mmol), HOBt.H₂O (97 mg, 0.72 mmol), and diisopropylethylamine (0.42 mL, 2.4 mmol) in anhydrous DMF (3 mL) at RT. The reaction was stirred at RT for 22.5 h, then was concentrated on the rotavap. The residue was reconcentrated from xylenes (to remove DMF), then was diluted with H₂O (2 mL). CHCl₃ extraction, drying (MgSO₄), concentration, and chromatography on silica gel (5% MeOH/CHCl₃) gave the title compound (265.7 mg, 92%) as an off-white solid.

b) (±)-7-[[[2-(Benzimidazolyl)methyl]methylamino]carbonyl]-4-(2-methoxyethyl)-3-oxo-2,3,4,5-tetrahydro-1H-1,4-benzodiazepine-2-acetic acid

1.0 N LiOH (0.66 mL, 0.66 mmol) was added to a solution of methyl (±)-7-[[[2-(benzimidazolyl)methyl]methylamino]carbonyl]-4-(2-methoxyethyl)-3-oxo-2,3,4,5-tetrahydro-1H-1,4-benzodiazepine-2-acetate (265.7 mg, 0.55 mmol) in THF (2.8 mL) and H₂O (2.1 mL) at RT. The light yellow solution was stirred at RT for 17 h, then was concentrated to dryness on the rotavap. The residue was dissolved in H₂O (2 mL), and the solution was neutralized with 1.0 N HCl (0.66 mL). The solid precipitate was collected by suction filtration and recrystallized from H₂O/CH₃CN to afford the title compound (147.0 mg, 55%).

### EXAMPLE 12

Preparation of (±)-7-[[[2-(4-Azabenzimidazolyl)methyl]methylamino]carbonyl]-4-( 2-methoxyethyl)-3-oxo-2,3,4,5-tetrahydro-1H-1,4-benzodiazepine-2-acetic acid

a) Methyl (±)-7-[[[2-(4-azabenzimidazolyl)methyl]methylamino]carbonyl]-4-(2-methoxyethyl)-3-oxo-2,3,4,5-tetrahydro-1H-1,4-benzodiazepine-2-acetate

EDC (115 mg, 0.60 mmol) was added to a solution of methyl (±)-7-carboxy-4-(2-methoxyethyl)-3-oxo-2,3,4,5-tetrahydro-1H-1,4-benzodiazepine-2-acetate (168.2 mg, 0.50 mmol), 4-aza-2-(methylaminomethyl)benzimidazole (0.62 mmol), HOBt.H₂O (81 mg, 0.60 mmol), and diisopropylethylamine (0.17 mL, 1.0 mmol) in anhydrous DMF (2.5 mL) at RT. The reaction was stirred at RT for 20 h, then was concentrated on the rotavap, and the residue was diluted with H₂O (2 mL). CHCl₃ extraction (3×5 mL), drying (MgSO₄). concentration, and reconcentration from xylenes (to remove DMF) left a light yellow oil. Chromatography on silica gel (10% MeOH/CHCl₃) gave the title compound (225.4 mg, 94%) as a colorless foam.

b) (±)-7-[[2-(4-Azabenzimidazolyl)methyl]methylamino]carbonyl]-4-(2-methoxyethyl)-3-oxo-2,3,4,5-tetrahydro-1H-1,4-benzodiazepine-2-acetic acid

1.0 N LiOH (0.56 mL, 0.56 mmol) was added to a solution of methyl (±)-7-[[[2-(4-azabenzimidazolyl)methyl]methylamino]carbonyl]-4-(2-methoxyethyl)-3-oxo-2,3,4,5-tetrahydro-1H-1,4-benzodiazepine-2-acetate (225.4 mg, 0.47 mmol) in THF (2.4 mL) and H₂O (1.8 mL) at RT. The solution was stirred at RT for 16.5 h, then was acidified with TFA (0.11 mL) and concentrated to dryness on the rotavap. ODS chromatography (step gradient: 12% CH₃CN/H₂O-0.1% TFA, then 20% CH₃CN/H₂O-0.1% TFA). concentration to a small volume, and lyophilization gave the title compound (167.2 mg, 55%) as a light yellow powder.

### EXAMPLE 13

Preparation of (±)-7-[[[(2-benzimidazolyl)methyl]amino]methyl]-4-methyl-3-oxo-2,3,4,5-tetrahydro-1H-1,4-benzodiazepine-2-acetic acid

a) Methyl (±)-7-[[[(2-benzimidazolyl)methyl]amino]methyl]-4-methyl-3-oxo-2,3,4,5-tetrahydro-1H-1,4-benzodiazepine-2-acetate

Methyl (±)-7-formyl-4-methyl-3-oxo-2,3,4,5-tetrahydro-1H-1,4-benzodiazepine-2-acetate (180 mg, 0.65 mmol) was suspended in anhydrous methanol, then sodium acetate (160 mg, 1.95 mmol), 2-(amino methyl)benzimidazole dihydrochloride (143 mg, 0.65 mmol) and 4 Å molecular sieves were added. After 30 min., sodium cyanoborohydride (45 mg, 0.71 mmol) was added in 2 portions over a period of 30 min. The reaction mixture was allowed to stir at RT overnight, then the methanol was removed under vacuum. The residue was diluted with CH₂Cl₂, and the solution was washed with saturated NaHCO₃. Drying (MgSO₄), concentration, and silica gel chromatography (90% CH₂Cl₂/9% methanol/1% NEt₃) gave the title compound (133 mg, 49%).

b) (±)-7-[[[(2-Benzimidazolyl)methyl]amino]methyl]-4-methyl-3-oxo-2,3,4,5-tetrahydro-1H-1,4-benzodiazepine-2-acetic acid

LiOH (14.6 mg, 0.34 mmol) was added at RT to a solution of methyl (±)-7-[[[(2-benzimidazoyl)methyl]amino]methyl]-4-methyl-3-oxo-2,3,4,5-tetrahydro-1H-1,4-benzodiazepine-2-acetate (133 mg, 0.31 mmol) in dioxane (3 mL) and H₂O (1 mL). The reaction mixture was stirred at RT overnight then the organic solvent was removed in vacuo. The aqueous residue was acidified with 1 M HCl solution (0.38 mL) to obtain a white solid which was purified by ODS chromatography (10% acetonitrile/H₂O-0.1% TFA) to afford the title compound (65 mg, 51%).

### EXAMPLE 14

Preparation of (±)-7-[[[(2-benzimidazolyl methyl]methylamino]carbonyl]-3-oxo-2,3,4,5-tetrahydro-1H-1,4-benzodiazepine-2-acetic acid

a) Methyl (±)-7-[[[(2-benzimidazolylmethyl methylamino]carbonyl]-3-oxo-2,3,4,5-tetrahydro-1H-1,4-benzodiazepine-2-acetate

Following the procedure of Example 8(b), except substituting methyl 7-carboxy-3-oxo-2,3,4,5-tetrahydro-1H-1,4-benzodiazepine-2-acetate for the methyl (±)-7-carboxy-4-methyl-3-oxo-2,3,4,5-tetrahydro-1H-1,4benzodiazepine-2-acetate, the crude title compound was prepared. Chromatography (silica gel, 7% MeOH/CH₂Cl₂) yielded the title compound (35%).

b) (±)-7-[[[(2-Benzimidazolyl)methyl]methylamino]carbonyl]-3-oxo-2,3,4,5-tetrahydro-1H-1,4-benzodiazepine-2-acetic acid

A solution of methyl (±)-7-[[[(2-benzimidazolyl)methyl]methylamino]carbonyl]-3-oxo-2,3,4,5-tetrahydro-1H-1,4-benzodiazepine-2-acetate (0.040 g, 0.09 mmol) in a mixture of methanol (7.0 mL), water (0.7 mL), and 1.0 M NaOH (0.7 mL) was kept 16 h at RT. Trifluoroacetic acid (0.5 mL) was added and the solvents were removed to give the crude product. Purification by semi-preparative HPLC (YMC ODS-AQ, 15:85; acetonitrile:water, 0.1% TFA) gave the title compound.

### EXAMPLE 15

Preparation of (2S)-7-[[[N-butyl-N-benzimidazol-2-yl)methyl]amino]carbonyl]-3-oxo-4-methyl-2,3,4,5-tetrahydro-1H-1,4-benzodiazepine-2-acetic acid

a) N-BOC-2-methylbenzimidazole

To a stirred mixture of 2-methylbenzimidazole (15 g, 113.5 mmol), triethylamine (12 g, 119.2 mmol), and DMAP (cat.) in dry CH₂Cl₂ (150 mL) was added (Boc)₂O. After 24 h, the mixture was concentrated. The residue was taken up in H₂O, stirred and filtered to give a white solid (26.3 g, 100%).

b) 1-BOC-2-bromomethylbenzimidazole

A mixture of N-BOC-2-methylbenzimidazole (13.40 mmol), N-bromosuccinimide (2.39 g, 13.40 mmol), and AIBN (0.5 g, 3.04 mmol) in CCl₄ (40 mL) was refluxed for 12h, then the mixture was cooled and filtered. The filtrate was concentrated and purified by chromatography (silica gel, 5% EtOAc/hexane) to give the title compound as a yellow oil (12.88 g, 77%).

c) 2-(1-Butylamino)methylbenzimidazole

To a stirred solution of 1-BOC-2-bromomethylbenzimidazole (2.00 g, 6.4 mmol) in dry THF (20 mL) was added n-butylamine (1.2 g, 15.4 mmol). After stirring at RT overnight, the mixture was concentrated. The residue was taken up in H₂O and extracted with CH₂Cl₂. The organic extracts were dried over MgSO₄ and concentrated to give a brown residue, which was dissolved in CH₂Cl₂ (15 mL) and treated with TFA (5 mL). The resulting mixture was stirred at RT overnight then was concentrated. The residue was taken up in H₂O, and the solution was neutralized with 2.5 N NaOH. CH₂Cl₂ extraction, drying (MgSO₄), concentration, and silica gel chromatography (2% MeOH/CH₂Cl₂) gave the title compound as a yellow oil (0.91 g, 70%).

d) Methyl-(S)-7-[[[N-(2-benzimidazolyl)methyl-N-(n-butyl)amino]carbonyl]-4-methyl-3-oxo-2,3,4,5-tetrahydro-1H-1,4-benzodiazepine-2-acetate

To a stirred mixture of 2-(1-butylamino)methylbenzimidazole (0.14 g, 0.6671 mmol), methyl (S)-7-carboxy-4-methyl-3-oxo-2,3,4,5-tetrahydro-1H-1,4-benzodiazepine-2-acetate (0.15 g, 0.5132 mmol), HOBT.H20 (0.083 g, 0.6158 mmol), and (i-Pr)₂NEt (0.133 g, 1.0263 mmol) in dry MeCN (5 mL) was added EDC (0.183 g, 0.6158 mmol). After stirring at RT overnight, the mixture was concentrated. The residue was taken up in H₂O and extracted with CH₂Cl₂. The combined organic layers were washed sequentially with saturated NaHCO₃ and brine, then were dried (MgSO₄) and concentrated to give the title compound as a yellow foam (0.232 g, 95%).

e) (S)-7[[[N-(2-Benzimidazolyl)methyl-N-(n-butyl)]amino]carbonyl]-4-methyl-3-oxo-2,3,4,5-tetrahydro-1H-1,4-benzodiazepine-2-acetic acid

Following the procedure in Example 5(b), methyl-(S)-7-[[[N-(2-benzimidazolyl)methyl-N-(n-butyl)amino]carbonyl]-4-methyl-3-oxo-2,3,4,5-tetrahydro-1H-1,4-benzodiazepine-2-acetate was saponified to give an off white solid. Trituration in hot EtOH gave the title compound as a white solid (0.15 g, 60%).

### EXAMPLE 16

Preparation of (S)-7-[[[N-(2-benzimidazolyl)methyl-N-(2-phenylethyl)]amino]carbonyl]-4-methyl-3-oxo-2,3,4,5-tetrahydro-1H-1,4-benzodiazepine-2-acetic acid

a) 2-(2-Phenylethylamino)methylbenzimidazole

Following the procedure of Example 15(c), except substituting 2-phenylethylamine for n-butylamine, the title compound (0.100 g, 3 1%) was prepared as a brown oil following silica gel flash chromatography (5% MeOH/CH₂Cl₂).

b) Methyl (S)-7-[[[N-(2-benzimidazolyl)methyl-N-(2-phenylethyl)]amino]carbonyl]-4-methyl-3 -oxo-2,3,4,5-tetrahydro-1H-1,4-benzodiazepine-2-acetate

Following the procedure of Example 15(d), except substituting 2-(2-phenylethylamino)methylbenzimidazole for 2-(1-butylamino)methylbenzimidazole, the title compound (0.195 g, 97%) was prepared as an off-white foam following silica gel flash chromatography (2-5% MeOH/CH₂Cl₂).

c) (S)-7-[[[N-(2-Benzimidazolyl)methyl-N-(2-phenylethyl)]amno]carbonyl]-4-methyl-3-oxo-2,3,4,5-tetrahydro-1H-1,4-benzodiazepine-2-acetic acid

Following the procedure of Example 5(b), methyl (S)-7-[[[N-(2-benzimidazolyl)methyl-N-(2-phenylethyl)]amino]carbonyl]-4-methyl-3-oxo-2,3,4,5-tetrahydro-1H-1,4-benzodiazepine-2-acetate was saponified. Recrystallization from EtOH gave the title compound (0.070 g, 40%) as an off white solid.

### EXAMPLE 17

Preparation of (S)-7-[[[2-(5,6-methylendioxybenzimidazolyl)methyl]methylamino]carbonyl]-4-methyl-3-oxo-2,3,4,5-tetrahydro-1H-1,4-benzodiazepine-2-acetic acid

a) 2-[[N-(Benzyloxycarbonyl)-N-methyl]aminomethyl]-5,6-methylenedioxybenzimidazole

Cbz-sarcosine (310.0 mg, 1.39 mmol) was dissolved in dry THF (10 mL) in a 250 mL roundbottom flask under argon. Isobutylchloroformate (0.2 mL, 1.54 mmol) was added, followed by Et₃N (0.25 mL, 1.80 mmol). The reaction was stirred at RT under argon for 30 min, then was cooled to −10° C. to −20° C. A solution of 1,2-dialino-4,5-methylenedioxybenzene (0.2 g, 1.314 mmol) in dry THF was added, and the reaction was allowed to warm to RT. After 18 h, the reaction was concentrated under vacuum. The white solid residue was dissolved in EtOAc, and the solution was washed with 1.0 N NaHCO₃. The organic layer was dried (MgSO₄), filtered, and concentrated under vacuum. The residue was dissolved in glacial AcOH and heated an oil bath set at 70° C. After 24 h, the reaction was concentrated under vacuum. The residue was reconcentrated from toluene, then was chromatographed on silica gel (1:1 CH₂Cl₂/Et₂O). The material obtained in this way (two components co-eluted) was redissolved in glacial AcOH and heated to 100° C. TLC of the reaction after 24 h still showed two products. Concentration and chromatography (silica gel, 1:1 CHCl₃/Et₂O) gave the title compound (145.0 mg, 32.7%).

b) 2-(Methylamino)methyl-5,6-methylenedioxybenzimidazole

2-[[N-(Benzyloxycarbonyl)-N-methyl]aminomethyl]-5,6-methylenedioxybenzimidazole (145.0 mg, 0.43 mmol) was dissolved in MeOH. and 10% Pd/C was added. The mixture was stirred briskly at RT under H₂ (balloon). After 4 h, the reaction was filtered through CELITE®, and the filtrate was concentrated under vacuum to afford the title compound (70.8 mg, 80.2%).

c) Methyl (S)-7-[[[2-(5,6-methylendioxybenzimidazolyl)methyl]methylamino]carbonyl3-4-methyl-3-oxo-2,3,4,5-tetrahydro-1H-1,4-benzodiazepine-2-acetate

EDC (76.2 mg, 0.40 mmol) was added to a solution of methyl (2S)-7-carboxy-4-methyl-3-oxo-2,3,4,5-tetrahydro-1H-1,4-benzodiazepine-2-acetate (0.35 mmol) and HOBt.H₂O (57.9 mg, 0.43 mmol) in dry DMF, and the reaction was stirred at RT. Diisopropylethylamine (0.150 mL, 0.86 mmol) was added, followed by a solution of 2-(methylamino)methyl-5,6-methylenedioxybenzimidazole (70.8 mg, 0.35 mmol) in dry DMF. The reaction was stirred at RT for 24 h, then was concentrated under vacuum. Chromatography (silica gel, step gradient, CHCl₃, 1:1 MeOH/CHCl₃) and rechromatography (2% MeOH/CHCl₃, 10% MeOH/CHCl₃) gave the title compound (102.5 mg, 61.1%).

d) (S)-7-[[[2-(5,6-Methylendioxybenzimidazolyl)methyl]methylamino]carbonyl]-4-methyl-3-oxo-2,3,4,5-tetrahydro-1H-1,4-benzodiazepine-2-acetic acid

Methyl (S)-7- [[[2-(5,6-methylendioxybenzimidazolyl)methyl]methylamino]carbonyl]-4-methyl-3-oxo-2,3,4,5-tetrahydro-1H-1,4-benzodiazepine-2-acetate (102.5 mg, 0.21 mmol) was dissolved in MeOH, and 1.0 N NaOH (0.5 mL, 0.5 mmol) was added. The reaction was stirred at RT for 48 h, then was neutralized with 1.0 N HCl. Concentration under vacuum left a residue which was diluted with water and allowed to stand at RT overnight. The resultant precipitate was collected by filtration and dried under high vacuum to yield the title compound (29.0 mg, 30%).

### EXAMPLE 18

Preparation of (S)-7-[[[2-(4-azabenzimidazolyl)methyl]methylamino]carbonyl]-4-methyl-3-oxo-2,3,4,5-tetrahydro-1H-1,4-benzodiazepine-2-acetic acid

a) 2-[[N-(Benzyloxycarbonyl)-N-methyl]aminomethyl]-4-azabenzimidazole

A solution of Cbz-sarcosine (5 g, 22.4 mmol) and Et₃N (4 mL, 28.76 mmol) in dry THF was cooled to 0° C. in an ice bath, and isobutylchloroformate (3.0 mL, 23.13 mmol) was added. The reaction was stirred at RT for 15 min. then was added to a solution of 2,3-diaminopyridine (2.5 g, 22.7 mmol) in dry THF at −25° C. The reaction was stirred at −20° C. for 30 min. then was allowed to warm to RT. After 24 h, the reaction was concentrated under vacuum. The residue was taken up in EtOAc and washed with 1.0 N NaHCO₃. The organic layer was dried (MgSO₄), filtered and concentrated under vacuum. The residue was dissolved in glacial AcOH (200 mL) and heated in an oil bath set at 109° C. After 20 h, the reaction was concentrated under vacuum, and the residue was reconcentrated from toluene. Chromatography (Silica gel, step gradient, CHCl₃, 3% MeOH/CHCl₃, 5% MeOH/CHCl₃) gave the title compound (2.2 g, 33%), which was recrystallized from Et₂O.

b) 2-(Methylamino)methyl-4-azabenzimidazole

2-[[N-(Benzyloxycarbonyl)-N-methyl]aminomethyl]-4-azabenzimidazole (551.3 mg, 1.86 mmol) was dissolved in MeOH, and 10% Pd/C was added. The mixture was stirred briskly at RT under H₂ (balloon). After 4 h, the reaction was filtered through CELITE®, and the filtrate was concentrated under vacuum to afford the title compound (420.1 mg, quantitative).

c) Methyl (S)-7-[[[2-(4-azabenzmidazolyl)methyl]methylamino]carbonyl]-4-methyl-3-oxo-2,3,4,5-tetrahydro-1H-1,4-benzodiazepine-2-acetate

EDC (309.1 mg, 1.61 mmol) was added to a solution of methyl (S)-7-carboxy-4-methyl-3-oxo-2,3,4,5-tetrahydro-1H-1,4-benzodiazepine-2-acetate (504.6 mg, 1.54 mmol), diisopropylethylamine (0.30 mL, 1.78 mmol), and HOBt H₂O (228.2 mg, 1.69 mmol) in dry DMF at RT. After 10 minutes, 2-(methylamino)methyl-4-azabenzimidazole (3.08 mmol) neutralized with diisopropylethylamine (0.600 mL) was added, and the reaction was stirred at RT. After 20 h, the solvents were evaporated under vacuum, and the residue was reconcentrated from toluene. Chromatography (silica gel, step gradient. CHCl₃, 5% MeOH/CHCl₃, 10% MeOH/CHCl₃) gave the title compound (326.8 mg, 48.6%).

d) (S)-7-[[[2-(4-Azabenzimidazolyl)methyl]methylamino]carbonyl]-4-methyl-3-oxo-2,3,4,5-tetrahydro-1H-1,4-benzodiazepine-2-acetate

1.0 N NaOH (2.0 mL, 2.0 mmol) was added to a solution of methyl (S)-7-[[[2-(4-azabenzimidazolyl)methyl]methylamino]carbonyl]-4-methyl-3-oxo-2,3,4,5-tetrahydro-1H-1,4-benzodiazepine-2-acetate (326.8 mg, 0.75 mmol) in MeOH (10 m) and H₂O (10 mL) at RT. After 26 h, the reaction was neutralized with 1.0 N HCl (2.0 mL, 2.0 mmol) and concentrated under vacuum. The residue was taken up in H₂O and the resultant white precipitate was collected on a sintered glass funnel, washed with H₂O and dried under vacuum to afford the title compound (218.1 mg, 69%) as a white powder.

### EXAMPLE 19

Preparation of (±)-7-[[[2-(4-azabenzimidazolyl)methyl]methylamino]carbonyl]-4-isopropyl-3-oxo-2,3,4,5-tetrahydro-1H-1,4-benzodiazepine-2-acetic acid

a) Methyl (±)-7-[[[2-(4-azabenzimidazolyl)methyl]methylamino]carbonyl]-4-isopropyl-3-oxo-2,3,4,5-tetrahydro-1H-1,4-benzodiazepine-2-acetate

Following the procedure of Example 18(c), except substituting methyl (±)-7-carboxy-4-isopropyl-3-oxo-2,3,4,5-tetrahydro-1H-1,4-benzodiazepine-2-acetate for the methyl (S)-7-carboxy-4-methyl-3-oxo-2,3,4,5-tetrahydro-1H- 1,4-benzodiazepine-2-acetate, the title compound (226 mg, 96%) was prepared.

b) (±)-7-[[[2-(4-Azabenzimidazoyl)methyl]methylamino]carbonyl]-4-isopropyl-3-oxo-2,3,4,5-tetrahydro-1H-1,4-benzodiazepine-2-acetic acid

1.0 N NaOH (1.5 mL, 1.5 mmol) was added to a solution of methyl (±)-7-[[[2-(4-azabenzimidazolyl)methyl]methylamino]carbonyl]-4-isopropyl-3-oxo-2,3,4,5-tetrahydro-1H-1,4-benzodiazepine-2-acetate (226.2 mg, 0.49 mmol) in MeOH (5 mL) and H₂O (5 mL) at RT. After 24 h, the reaction was neutralized with 1.0 N HCl and the solvents were evaporated under vacuum. ODS chromatography (0.1% TFA/H₂O, followed by 20% CH₃CN/H₂O-0% TFA), concentration, and reconcentration from toluene left a residue, which was redissolved in H₂O. Lyophilization gave impure title compound (181.9 mg) as a white powder, which was repurified by ODS chromatography (10% CH₃CN/H₂O-0.1% TFA, followed by 20% CH₃CN/H₂O-0.1% TFA). Concentration and reconcentration from toluene left a residue, which was dissolved in MeOH and precipitated with Et₂O. The precipitate was collected on a sintered glass funnel and dried in a vacuum desiccator to afford the title compound (65.5 mg).

### EXAMPLE 20

Preparation of (S)-7-[[[2-(4-aza-5-methylbenzimidazolyl)methyl]methylamino]carbonyl]-4-methyl-3-oxo-2,3,4,5-tetrahydro-1H-1,4-benzodiazepine-2-acetic acid

a) 2-Amino-6-methyl-3-nitropyridine

2-Amino-6-picoline (5.1 g, 47.1 mmol) was weighed into a 500 mL round bottom flask, and the flask was cooled to −30° C. Concentrated H₂SO₄ (20 mL) was added, which caused some fuming to occur. Concentrated HNO₃ (10 mL, 160 mmol) was then added dropwise slowly. The reaction was allowed to warm to RT over 30 min, then was heated in an oil bath set at 80° C. After 90 min, the reaction was removed from the heating bath. and ice was added. 6.25 N NaOH (150 mL, 937.5 mmol) was added slowly, and the resulting yellow precipitate was collected on a sintered glass funnel. Drying in a vacuum desiccator gave the title compound (1.7 ,24%).

b) 2,3-Diamino-6-methylpyridine

2-Amino-6-methyl-3-nitropyridine (754 mg, 4.92 mmol) was suspended in MeOH, and 10% Pd/C was added. The mixture was stirred briskly at RT under H₂ (balloon). After 4 h, the reaction was filtered through CELITE®, and the filtrate was concentrated under vacuum to afford the title compound (677. mg, quantitative).

c) 2-[[N-(Benzyloxycarbonyl)-N-methyl]aminomethyl]-5-methyl4-azabenzimidazole

A solution of Cbz-sarcosine (1.8 g, 7.85 mmol) in dry THF at RT was treated with isobutylchloroformate (1.25 mL, 9.64 mmol), followed by Et₃N (3.0 mL. 21.57 mmol). After 30 min, a solution of 2,3-diamino-6-methylpyridine (882 mg, 7.16 mmol) In dry THF was added, and the reaction was stirred at RT. After 3 d. the reaction was concentrated under vacuum. The residue was taken up in EtOAc and washed with 1.0 N NaHCO₃. The organic layer was dried (MgSO₄), filtered, concentrated under vacuum, and reconcentrated from toluene. The residue was dissolved in glacial AcOH (100 mL) and heated in an oil bath set at 110° C. After 24 h, the reaction was concentrated under vacuum, and the residue was reconcentrated from toluene. Chromatography (silica gel, step gradient, CHCl₃, 2% MeOH/CHCl₃, 3% MeOH/CHCl₃) gave the title compound (1.0 g, 46.6%).

d) 2-(Methylamino)methyl-5-methyl-4-azabenzimidazole

2-[[N-(Benzyloxycarbonyl)N-methyl]aminomethyl]-5-methyl-4-azabenzimidazole (1.0347 g, 0.33 mmol) was dissolved in MeOH, and 10% Pd/C was added. The mixture was stirred briskly at RT under H₂ (balloon). After 20 h, the reaction was filtered through CELITE®, and the light yellow filtrate was concentrated under vacuum to afford the title compound (678.9 mg, quantitative) as a reddish colored material.

e) Methyl (S)-7-[[[2-(4-aza-5-methylbenzimidazolyl)methyl]methylamino]carbonyl]-4-methyl-3-oxo-2,3,4,5-tetrahydro-1H-1,4-benzodiazepine-2-acetate

EDC (212.7 mg, 1.11 mmol) was added to a solution of methyl (S)-7-carboxy-4-methyl-3-oxo-2,3,4,5-tetrahydro-1H-1,4-benzodiazepine-2-acetate (293.5 mg, 0.93 mmol), diisopropylethylamine (0.30 mL, 1.72 mmol), and HOBt.H₂O (143.5 mg, 1.06 mmol) in dry DMF at RT. After 30 minutes, a solution of 2-(methylamino)methyl-5-methyl-4-azabenzimidazole (190.7 mg, 1.08 mmol) in dry DMF was added. The reaction was stirred at RT for 24 h, then was concentrated under vacuum, and the residue was reconcentrated from toluene. Chromatography (silica gel, step gradient, CHCl₃, 3% MeOH/CHCl₃, 5% MeOH/CHCl₃) gave the title compound (265 mg, 63%).

f) (S)-7-[[[2-(4-Aza-5-methylbenzimidazolyl)methyl]methylamino]carbonyl]-4-methyl-3-oxo-2,3,4,5-tetrahydro-1H-1,4-benzodiazepine-2-acetic acid

1.0 N NaOH (2.0 mL, 2.0 mmol) was added to a solution of methyl (S)-7-([[2-(4-aza-5-methylbenzimidazolyl)methyl]methylamino]carbonyl]-4-methyl-3-oxo-2,3,4,5-tetrahydro-1H-1,4-benzodiazepine-2-acetate (264.7 mg, 0.59 mmol) in MeOH (10 mL) and H₂O (10 mL) at RT. After 20 h, the reaction was neutralized with 1.0 N HCl (2.0 mL) and the solvents were evaporated under vacuum. The crude material was precipitated from water to give the title compound (49.8 mg).

### EXAMPLE 21

Preparation of (S)-7-[[[(2-benzimidazolyl)methyl]methylamino]carbonyl]-3-oxo-4-(2-phenylethyl)-2,3,4,5-tetrahydro-1H-1,4-benzodiazepine-2-acetic acid

a) Methyl (S)-7-carboxy-3-oxo-4(2-phenylethyl)-2,3,4,5-tetrahydro-1H-1,4-benzodiazepine-2-acetate

To a solution stirred under argon at RT of methyl (±)-7-carboxy-3-oxo-4-(2-phenylethyl)-2,3,4,5-tetrahydro-1H-1,4-benzodiazepine-2-acetate (9.0 g, 23 mmol) in CH₃CN (100 mL) was added diazabicycloundecene (4.6 g, 30 mmol), followed by benzyl bromide (20 g, 116 mmol). The resulting solution was stirred for 1 h, then was concentrated. The residue was partitioned between 1.0 N HCl and EtOAc, and the layers were separated. The organic layer was washed with brine, dried (MgSO₄), and concentrated. The residue was purified by chromatography (silica gel, CH₂Cl₂) to give a pale yellow oil (7 g). Preparative HPLC (Whelk O-1, 50:50:1 hexane:CHCl₃:CH₃OH) gave an oil which was 97% of the desired (S)-enantiomer. Removal of racemate by crystallization (EtOAc) gave a colorless oil (3.2 g, 98% ee). This material was placed in a 500 mL Parr hydrogenation vessel with CH₃OH (30 mL) and 10% Pd/C (0.45 g), and the mixture was shaken under H₂ (50 psi) for 6 h. The reaction mixture was then filtered and the filtrate was concentrated to give the title compound as a colorless foam (2.1 g, 47%).

b) Methyl (S)-7-[[[(2-benzimidazolyl)methyl]methylamino]carbonyl]-3oxo-4-(2-phenylethy-1)-2,3,4,5-tetrahydro-1H-1,4-benzodiazepine-2-acetate

Methyl (S)-7-carboxy-3-oxo4-(2-phenylethyl)-2,3,4,5-tetrahydro-1H-1,4-benzodiazepine-2-acetate was coupled with 2-(methylamino)methylbenzimidazole according to the procedure of Example 2(a). Purification by chromatography on silica gel (1%-5% CH₃OH/CH₂Cl₂) gave the title compound (2.85 g, 99%) as a colorless foam.

c) (S)-7-[[[(2-Benzimidazolyl)methyl]methylamino]carbonyl]-3-oxo-4-(2-phenylethyl)-2,3 ,4,5-tetrahydro-1H-1,4-benzodiazepine-2-acetic acid

Methyl (S)-7-[[[(2-benzimidazolyl)methyl]methylaminocarbonyl]-3-oxo-4-(2-phenylethyl)-2,3,4,5-tetrahydro-1H-1,4-benzodiazepine-2-acetate was saponified according to the procedure of Example 5(b) to give the title compound (1.8 g, 66%) as a white solid.

### Example 22

Preparation of (±)-7-[[[2-(benzimidazolyl)methyl]amino]carbonyl-4-[2-(3,4-methylenedioxyphenyl)ethyl]-3-oxo-2,3,4,5-tetrahydro-1H-1,4-benzodiazepine2-acetic acid

a) Methyl (±)-7-[[[2-(benzimidazolyl)methyl]amino]carbonyl-4-[2-(3,4-methylenedioxyphenyl)ethyl]-3-oxo-2,3,4,5-tetrahydro-1H-1,4-benzodiazepine-2-acetate

Methyl (±)-7-carboxy-4-[2-(3,4-methylenedioxyphenyl)ethyl]-3-oxo-2,3,4,5-tetrahydro-1H-1,4-benzodiazepine-2-acetate was coupled with 2-(aminomethyl)benzimidazole dihydrochloride hydrate according to the procedure of Example 2(a). Purification by chromatography (silica gel, 1%-5% CH₃H/CH₂Cl₂) followed by recrystallization (CH30H/EtOAc) gave the title compound as a tan solid (59%).

b) (±)-7-[[[2-(Benzimidazolyl)methyl]amino]carbonyl-4-[2-(3,4-methylenedioxyphenyl)ethyl]-3-oxo-2,3,4,5-tetrahydro-1H-1,4-benzodiazepine-2-acetic acid

Methyl (±)-7-[[[2-(benzimidazoyl)methyl]amino]carbonyl-4-[2-(3,4-methylenedioxyphenyl)ethyl]-3-oxo-2,3,4,5-tetrahydro-1H-1,4-benzodiazepine-2-acetate was saponified according to the procedure of Example 5(b) to give the title compound (84%) as a white solid.

### EXAMPLE 23

Preparation of (±)-2,3,4,5-Tetrahydro-3-oxo-4-(phenylethyl)-7-[[[2-(2-(pyridinyl)amino]ethyl]amino]carbonyl]-1H-1,4-benzodiazepine-2-acetic acid

a) Methyl (±)-2,3,4,5-tetrahydro-3-oxo4-(phenylethyl)-7-[[[2-[2-(pyridinyl)amino]ethyl]amino]carbonyl]-1H-1,4-benzodiazepine-2-acetate

Methyl (±)-7-carboxy-2,3,4,5-tetrahydro-3-oxo4-phenylethyl-1H-1,4-benzodiazepine-2-acetate (2 mmol), EDC (0.38 g, 2 mmol), HOBT.H₂O and the compound of Preparation 4(c) (1.65 mmol) were combined and stirred at RT overnight. The mixture was concentrated, and the residue was treated with 5% Na₂CO₁ and extracted with CH₂Cl₂. The combined organic extracts were washed with H₂O, dried (MgSO₄) and concentrated. The residue was chromatographed to yield the title compound as a white foam.

b) (±)-2,3,4,5-Tetrahydro-3-oxo-4-(phenylethyl )-7-[[[-2-[2-pyridinyl)amino]ethyl]amino]carbonyl]-1H-1,4-benzodiazepine-2-acetic acid

The compound of Example 23(a) (0.16 g, 0.4 mmol) was dissolved in CH₃OH (10 mL) and THF (1 mL), and treated with 1 N NaOH (0.5 mL). The mixture was stirred overnight, concentrated, and the residue was dissolved in H₂O and extracted with CH₂Cl₂, The pH of the aqueous phase was adjusted to 5.5-6 with dilute HCl, and the solid which formed was filtered, washed with H₂O and Et₂O, and dried to give the title compound.

### EXAMPLE 24

Preparation of (±)-8-[3-(2-pyridylamino)-1-propyloxy]-3-oxo-2,3,4,5-tetrahydro-1H-2-benzazepine-4-acetic acid

a) Methyl (±)-8-[3-[2-(N-oxopyridyl)amino]-1-propyloxyl-3-oxo-2,3,4,5-tetrahydro-1H-2-benzazepine4-acetate

A solution of 2-[(3-hydroxy-1-propyl)amino]pyridine-N-oxide (1.4 g, 8 mmol) in anhydrous DMF (8 mL) was added dropwise to a solution of methyl (±)-8-hydroxy-3-oxo-2,3,4,5-tetrahydro-1H-²-benzazepine-4-acetate (1.7 g, 7 mmole), triphenylphosphine (2.76 g,11 mmol), and diethyl azodicarboxylate (2.33 mL, 14 mmole) in anhydrous DMF (4 mL) and dry THF (10 mL) at RT under argon. The resulting solution was stirred for 18 hr. then was concentrated under vacuum. Silica gel chromatography (2%-10% CH₃0H/CH₂Cl₂) to give the title compound (1.2g).

b) Ethyl (±)-8-(3-(2-pyridylamino)-1-propyloxy]-3-oxo-2,3,4,5-tetrahydro-1H-2-benzazepine-4-acetate

A mixture of methyl (±)-8-[3-[2-(N-oxopyridyl)amino]-1-propyloxy]-3-oxo-2,3,4,5-tetrahydro-1H-2-benzazepine-4-acetate (1.2 g, 3 mmol), 1.2 g 10% palladium on charcoal (1.2 g), cyclohexene (3 mL, 15 mmol), and ethanol (20 mL) was heated at reflux for 18 hr. The mixture was filtered and the filtrate was concentrated. The residue was purified by chromatography on silica gel (2%-5% CH₃OH/CH₂Cl₂)to give the title compound (0.72 g, 64%) as a white foam.

c) (±)-8-[3-(2-pyridylamino)-1-propyloxy]-3-oxo-2,3,4,5-tetrahydro-1H-2-benzazepine-4-acetic acid

A mixture of ethyl (±)-8-[3-(2-pyridylamino)-1-propyloxy]-3-oxo-2,3,4,5-tetrahydro-1H-2-benzazepine-4-acetate (0.7 g, 2 mmol), lithium hydroxide monohydrate (0. 12 g, 3 mmol), 5 mL THF (5 mL), H₂O (5 mL), and MeOH (2 mL) was stirred at room temperature for 18 hr, then was then concentrated. The residue was partitioned between ethyl acetate and water, and the layers were separated. The aqueous phase was cautiously brought to pH 4 with 3 N HCl and allowed to stand. The resulting crystals were collected by filtration and dried to give the title compound (0.4 g, 65%) as a tan solid.

### EXAMPLE 25

Preparation of (±)-8-[3-[(2-imidazolin-2-yl)amino]-1-propyloxyl-2-methyl-3-oxo-2,3,4,5-tetrahydro-1H-2-benzazepine-4-acetic acid

a) Preparation of methyl(±)-8-[3-(4-nitrobenzyloxycarbonylamino)-1-propyloxy]-2-methyl-3-oxo-2,3,4,5-tetrahydro-1H-2-benzazepine-4-acetate

According to the procedure of Example 24(a), except substituting 3-(4-nitrobenzyloxycarbonylamino)-1-propanol for the 2-[(3-hydroxy-1-propyl)amino]pyridine-N-oxide, and substituting methyl (±)-8-hydroxy-2-methyl-3-oxo-2,3,4,5-tetrahydro-1H-2-benzazepine-4-acetate for the methyl (±)-8-hydroxy-3-oxo-2,3,4,5-tetrahydro-1H-2-benzazepine-4-acetate, the title compound was prepared as a colorless oil.

b) Methyl (±)-8-[3-amino-1-propyloxy]-2-methyl-3-oxo-2,3,4,5-tetrahydro-1H-2-benzazepine-4-acetate

A mixture of methyl (±)-8-[3-(4-nitrobenzyloxycarbonylamino)-1-propyloxy]-2-methyl-3-oxo-2,3,4,5-tetrahydro-1H-2-benzazepine-4-acecate (1.4 g, 3 mmol), 10% palladium on charcoal (0.55g, 0.6 mmol), and EtOH (20 mL) was stirred at RT under a balloon of hydrogen. After 18 hr, the mixture was filtered and the filtrate was concentrated to give the title compound (0.89 g, 99%) as a tan solid.

c) Methyl (±)-8-(3-[(2-imidazolin-2-yl)amino]-1-propyloxy]-2-methyl-3-oxo-2,3,4,5-tetrahydro-1H-2-benzazepine-4-acetate

A mixture of methyl (±)-8-[3-amino-1-propyloxy]-2-methyl-3-oxo-2,3,4,5-tetrahydro-1H-2-benzazepine-4-acetate (0.3 g, 1 mmol), 2-methylthioimidazoline (0.46g, 2 mmol), diisopropylethylamine (0.42 mL, 2 mmol) and dimethylacetamide (3 mL) was heated to 100° C. under argon. After 2 hr, the reaction was concentrated under vacuum and the residue was partitioned between CHCl₃ and H₂O. The organic phase was dried (MgSO₄) and concentrated, and the residue was purified by preparative HPLC to give the title compound (0.24g, 51%) as a yellow oil.

d) (±)-8-[3-[(2-Imidazolin-2-yl)amino]-1-propyloxy]-2-methyl-3-oxo-2,3,4,5-tetrahydro-1H-2-benzazepine4-acetic acid

Methyl(±)-8-[3-[(2-imidazolin-2-yl)amino]- 1 -propyloxy]-2-methyl-3-oxo-1,3,4,5-tetrahydro-1H-2-benzazepine-4-acetate was saponified according to the procedure of Example 24(c). Purification by preparative HPLC gave the title compound as a white solid.

### EXAMPLE 26

Preparation of (±)-8-[3-[(2-diazepin-2-yl)amino]-1-propyloxy]-2-methyl-3-oxo-2,3,4,5-tetrahydro-1H-2-benzazepine-4-acetic acid

a) Methyl (±)-8-[3-[(2-diazepin-2-yl)amino]-1-propyloxy]-2-methyl-3-oxo-2,3,4,5-tetrahydro-1H-2-benzazepine-4-acetate

According to the procedure of Example 25(c), except substituting 2-methylthio-1,3-diazepine for the 2-methylthioimidazoline, the title compound was prepared.

b) (±)-8-(3-[(2-Diazepin-2-yl)amino]-1-propyloxy]-2-methyl-3-oxo-2,3,4,5-tetrahydro-1H-2-benzazepine-4-acetic acid

According to the procedure of Example 25(d), except substituting methyl (±)-8-[3-[(2-diazepin-2-yl)amino]-1-propyloxy]-2-methyl-3-oxo-2,3,4,5-tetrahydro-1H-2-benzazepine-4-acetate for the methyl(±)-8-[3-[(2-imidazolin-2-yl)amino]-1-propyloxy]-2-methyl-3-oxo-2,3,4,5-tetrahydro-1H-2-benzazepine-4-acetate the title compound was prepared.

### EXAMPLE 27

Preparation of (±)-8-[2-(2-aminothiazol-4-yl)-1-ethoxy]-2-methyl-3-oxo-2,3,4,5-tetrahydro-1H-2-benzazepine-4-acetic acid

a) Methyl (±)-8-[2-(2-aminothiazol4-yl)-1-ethoxy]-2-methyl-3-oxo-2,3,4,5-tetrahydro-1H-2-benzazepine-4-acetate

According to the procedure of Example 24(a), except substituting 2-(2-aminothiazol-4-yl)ethanol (WO 95/32710) for the 2-[(3-hydroxy-1-propyl)amino]pyridine-N-oxide, the title compound was prepared as white foam.

b) (±)-8-[2-(2-Aminothiazol-4-yl)-1-ethoxy]-2-methyl-3-oxo-2,3,4,5-tetrahydro-1H-2-benzazepine-4-acetic acid

According to the procedure of Example 24(c) except substituting methyl (±)-8-[2-(2-aminothiazol-4-yl)-1-ethoxy-2-methyl-3-oxo-2,3,4,5-tetrahydro-1H-2-benzazepine-4-acetate for the ethyl (±)-8-[3-(2-pyridylamino-1-propyloxy)-3-oxo-2,3,4,5-tetrahydro-1H-2-benzazepine-4-acetate, the title compound was prepared as a white solid.

### EXAMPLE 28

Preparation of (S)-3-oxo-8-[3-(pyridin-2-ylamino)-1-1-propyloxyl-2-(2-phenylethyl)-2,3,4,5-tetrahydro-1H-2-benzazeoine-4-acetic acid

a) Methyl (S)-3-oxo-8-[3-(1-oxopyridin-2-ylamino)-1-propyloxy]-2-(2-phenylethyl)-2,3,4,5-tetrahydro-1H-2-benzazepine-4-acetate

A solution of 2-[(3-hydroxy-1-propyl)aminopyridine-N-oxide (336 mg, 2.0 mmole) and diethyl azodicarboxylate (0.3 mL, 2.0 mmole) in anhydrous DMF (10 mL) was added to a solution of methyl (S)-8-hydroxy-3-oxo-2-(2-phenylethyl)-2,3,4,5-tetrahydro-1H-2-benzazepine4-acetate (350 mg, 1.0 mmole) and triphenylphosphine (525 mg, 2.0 mmole) in anhydrous DMF (10 mL) at RT. After 24 hr the mixture was concentrated. Flash silica gel chromatography (gradient: EtOAc (500 mL) then 5% MeOH/CHCl3) gave the title compound as an orange foam (288 mg, 57%).

b) Methyl (S)-3-oxo-8-[3-(pyridin-2-ylamino)-1-propyloxy]-2-(2-phenylethyl)-2,3,4,5-tetrahydro-1H-2-benzazepine 4-acetate

A mixture of methyl (S)-3-oxo-8-[3-(1-oxopyridin-2-ylamino)-1-propyloxy]-2-(2-phenylethyl)-2,3,4,5-tetrahydro-1H-2-benzazepine-4-acetate (288 mg, 0.57 mmole), cyclohexene (0.6 mL, 5.8 mmole), 10% Pd/C (62 mg, 0.58 mmole), and 2-propanol (6 mL) was heated to reflux under argon. After 31 hr the mixture was hot filtered through a pad of CELITE®, the filter pad was washed with hot 1:1 MeOH/CHCl₃ (200 mL), and the filtrate was concentrated. Flash silica gel chromatography (5% MeOH/CHCl₃) followed by a second flash silica gel chromatography (50% THF/cyclohexane) gave the title compound as an off-white foam (133 mg, 48%).

c) (S)-3-Oxo-8-[3-(pyridin-2-ylamino)-1-propyloxy]-2-(2-phenylethyl)-2,3,4,5-tetrahydro-1H-2-benzazepine-4-acetic acid

1.0 N LiOH (0.3 mL, 0.3 mmole) was added to a solution of methyl (S)-3-oxo-8-[3-(pyridin-2-ylamino)-1-propyloxy]-2-(2-phenylethyl)-2,3,4,5-tetrahydro-1H-2-benzazepine-4-acetate (133 mg, 0.27 mmole) in THF (1.5 mL) and H₂O (1.2 mL) at 0° C. The mixture was allowed to warm to RT over 18 hr. The mixture was washed with Et₂O (2×5 mL) then a mild vacuum was applied to remove residual organic solvents. The aqueous layer was passed through a 0.45 μm Acrodisk filter, then was carefully acidified to pH 6 using 10% HCl in H₂O at 0° C. The precipitate was collected, washed with H₂O, and dried under vacuum at 50° C. to give the title compound as a white solid (62 mg, 48%).

### EXAMPLE 29

Preparation of (S)-8-[2-[6-(methylamino)pyridin-2-yl]ethoxy]-3-oxo-2-(2-phenylethyl)-2,3,4,5-tetrahydro-1H-2-benzazepine-4-acetic acid

a) Methyl (S)-8-[2-[6-( methylamino)pyridin-2-yl]ethoxy]-3-oxo-2(2-phenylethyl)-2,3,4,5-tetrahydro-1H-2-benzazepine-4-acetate

Diisopropyl azodicarboxylate (0.3 mL, 1.5 mmole) was added to a solution of methyl (S)-8-hydroxy-3-oxo-2-(2-phenylethyl)-2,3,4,5-tetrahydro-1H-2-benzazepine-4-acetate (350 mg, 1.0 mmole), 6-(methylamino)-2-pyridylethanol (228 mg, 1.5 mmole), and triphenylphosphine (393 mg, 1.5 mmole) in anhydrous THF (10 mL) at 0° C. The mixture was allowed to warm to RT over 72 hr then was concentrated. Flash silica gel chromatography (50% EtOAc/hexanes) followed by a second flash silica gel chromatography (25% EtOAc/hexanes) gave the title compound as a white foam (250 mg, 51%).

b) (S)-8-2-[6-(Methylamino)pyridin-2-yl]ethoxy]-3-oxo-2-(2-phenylethyl)-2,3,4,5-tetrahydro-1H-2-benzazepine-4-acetic acid

1.0N LiOH (0.62 mL, 0.62 mmole) was added to a solution of methyl (S)-8-[2-[6-(methylamino)pyridin-2-yl]ethoxy]-3-oxo-2-(2-phenylethyl)-2,3,4,5-tetrahydro-1H-2-benzazepine-4-acetate (250 mg, 0.51 mmole) in THF (2.5 mL) and H₂O (1.9 mL) at 0° C., and the reaction was allowed to stir at RT for 18 hr. The mixture was washed with Et₂O (2×5 mL) then a mild vacuum was applied to remove residual organic solvents. The aqueous layer was passed through a 0.45 μm Acrodisk filter then was carefully acidified to pH 6 using 10% HCl in H₂O at 0° C. The precipitate was collected, washed with H₂O, and dried under vacuum at 50° C. to give the title compound (134 mg, 55%) as a white solid.

### EXAMPLE 30

Preparation of (±)-10,11-dihydro-3-[4-(pyridin-2-ylamino)-1-butyl]-5H-dibenzo[a,d]cycloheptene-10-acetic acid

a) 4-(2-Tetrahydropyranyloxy)-1-tributylstannyl-1-butyne

A solution of n-butyllithium in hexanes (1.6 M, 18.8 mL, 30 mmole) was added in a stream over 2 min to a solution of 2-(3-butynyloxy)tetrahydro-2H-pyran (4.7 mL, 30 mmole) in dry THF (60 mL) at 0° C. under argon. After 0.5 hr, tributyltin chloride (8.1 mL, 30 mmole) was added all at once, and the reaction was warmed to RT. After 3 hr, the reaction was diluted with hexanes (300 mL) and washed sequentially with H₂O (2×60 mL), 10% KF (2×30 mL), and saturated brine (60 mL). Drying (Na₂SO₄), concentration, and silica gel chromatography (3% EtOAc/hexanes) gave the title compound (3.58 g, 27%) as a nearly colorless oil.

b) Ethyl (±)-10,11-dihydro-3-[4-(2-tetrahydropyranyloxy)-1-butyn-1-yl]-5H-dibenzo[a,d]cycloheptene-10-acetate

A mixture of ethyl (±)-10,11-dihydro-3-(trifluoromethanesulfonyloxy)-5H-dibenzo[a,d]cycloheptene-10-acetate (1.34 g, 3.13 mmole), 4-(2-tetrahydropyranyloxy)-1-tributylstannyl-1-butyne (1.66 g, 3.76 mmole), LiCl (398 mg, 9.39 mmole), bis(triphenylphosphine)palladium dichloride (110 mg, 0.094 mmole), and anhydrous dioxane (31 mL) was heated at reflux under argon. After 1.5 hr, the reaction was concentrated to remove most of the dioxane, and the residue was taken up in Et₂O (100 mL). 10% KF (50 mL) was added and the mixture was stirred briskly for 0.5 hr. The aqueous layer was removed and the Et₂O layer was filtered through a mixture of CELITE® and MgSO₄. The filtrate was concentrated and the residue was chromatographed on silica gel (10% EtOAc/hexanes) to afford the title compound (1.12 g. 83%) as a pale yellow oil.

c) Ethyl (±)-10,11-dihydro-3-[4-(2-tetrahydropyranyloxy)-1-butyl)-5H-dibenzo[a,d]cycloheptene-10-acetate

A mixture of ethyl (±)-10,11-dihydro-3-[4-(2-tetrahydropyranyloxy)-1-butyn-1-yl]-5H-dibenzo[a,d]cycloheptene-10-acetate (1.2 g, 2.77 mmole), 10% Pd/C (0.3 g, 0.28 mmole), and EtOAc (28 mL) was shaken at RT under hydrogen (50 psi) on a Parr apparatus. After 3 hr, the reaction was filtered through CELITE® and the filtrate was concentrated. Silica el chromatography (10% EtOAc/hexanes) gave the title compound (1.06 g, 88%) as a colorless oil.

d) Ethyl (±)-10,11-dihydro-3-(4-hydroxy-1-butyl)-5H-dibenzo[a,d]cycloheptene-10-acetate

A solution of ethyl (±)-10,11-dihydro-3-[4-(2-tetrahydropyranyloxy)-1-butyl]-5H-dibenzo[a,d]cycloheptene-10-acetate (456.0 mg, 1.04 mmole) and p-toluenesulfonic acid monohydrate (60 mg, 0.3l mmole) in absolute EtOH (10 mL) was stirred at RT. After 2 hr, the reaction was quenched with 5% NaHCO₃ (1 ml) and concentrated to remove the EtOH. The residue was diluted with H₂O (2 mL) and extracted with CH₂Cl₂. Drying (MgSO₄), concentration, and silica el chromatography (1:1 EtOAc/hexanes) gave the title compound (342.4 mg, 93%) as a colorless oil.

e) Ethyl (±)-10,11-dihydro-3-[4-(N-phthalidimido)-1-butyl]-5H-dibenzo[a,d]cycloheptene-10-acetate

Diethyl azodicarboxylate (0.2 mL, 1.26 mmole) was added dropwise to a solution of ethyl (±)- 10,11 -dihydro-3-(4-hydroxy-1-butyl)-5H-dibenzo[a,d]cycloheptene-10-acetate (0.37 g, 1.05 mmole), triphenylphosphine (0.33 g, 1.26 mmole), and phthalimide (0.19 g, 1.26 mmole) in anhydrous THF (10 mL) at RT under argon. After 23 hr, the reaction was concentrated on the rotavap. Silica gel chromatography (30% EtOAc/hexanes) gave the title compound (0.35 g, 70%) as a colorless oil.

f) Ethyl (±)-10,11-dihydro-3-(4-amino-1-butyl)-5H-dibenzo[a,d]cycloheptene-10-acetate

Hydrazine monohydrate (0.11 g, 2.18 mmole) was added to a solution of ethyl (±)-10,11-dihydro-3-[4-(N-phthalidimido)-1-butyl]-5H-dibenzo[a,d]cycloheptene-10-acetate (0.35 g, 0.73 mmole) in absolute EtOH (10 mL) and toluene (2 mL) at RT. The reaction was stirred at RT for 17 hr, then was filtered, and the filter pad was washed with toluene. Concentration on the rotavap gave the title compound (0.23 g, 90%) as a colorless solid.

g) Ethyl (±)-10,11-dihydro-3-[4-(1-oxopyridin-2-ylamino)-1-butyl]-5H-dibenzo[a,d]cycloheptene-10-acetate

A mixture of 2-chloropyridine-N-oxide hydrochloride (0.31 g, 1.88 mmole), ethyl (±)-10.11-dihydro-3-(4-amino-1-butyl)-5H-dibenzo[a,d]cycloheptene-10-acetate (0.212 g, 0.63 mmole), and NaHCO₃ (0.26 g, 3.13 mmole) in tert-amyl alcohol (6 mL) was heated at reflux for 21 hr. The reaction mixture was diluted with CH₂Cl₂ (100 mL) and filtered, and the filtrate was concentrated on the rotavap. Silica gel chromatography (1:9:5 MeOH/CH₂Cl₂/EtOAc) gave the title compound (82 mg, 30%) as a yellow oil.

h) Ethyl (±)-10,11-dihydro-3-[4-(pyridin-2-ylamino)-1-butyl]-5H-dibenzo[a,d]cycloheptene-10-acetate

A mixture of ethyl (±)-10,11-dihydro-3-[4-(1-oxopyridin-2-ylamino)-1-butyl]-5H-dibenzo[a,d]cycloheptene-10-acetate (0.07 g, 0.16 mmole), 10% Pd/C (0.08 g, 0.075 mmole), cyclohexene (0.16 mL, 1.6 mmole), and isopropanol (4 mL) was heated at reflux under argon for 14 hr, then the catalyst was removed by filtration through CELITE®. The filter pad was washed with isopropanol and MeOH, and the filtrate was concentrated on the rotavap to give the title compound (0.046 g, 69%) as a clear oil.

i) Ethyl (±)-10,11-dihydro-3-[4-(pyridin-2-ylamino)-1-butyl]-5H-dibenzo[a,d]cycloheptene-10-acetic acid

A mixture of ethyl (±)-10,11-dihydro-3-[4-(pyridin-2-ylamino)-1-butyl]-5H-dibenzo[a,d]cycloheptene-10-acetate (46 mg, 0.11 mmole) and 1.0 N LiOH (0.66 mL, 0.66 mmole) in THF (3 mL) and H₂O (3 mL) was stirred at RT. After 24 hr, the reaction mixture was concentrated on the rotavap, and the residue was diluted with H₂O (5 mL). The solution was cooled in an ice bath, and 1.0 N AcOH was added slowly to give a white precipitate. Chromatography on C-18 YMC (45% CH₃CN/H₂O containing 0.1% TFA) gave the title compound (13 mg, 21%) as a white solid.

### EXAMPLE 31

Preparation of (R)-10,11-dihydro-3-[3-(pyridin-2-ylamino)-1-propyloxy]-5H-dibenzo[a,d]cycloheptene-10-acetic acid

a) Ethyl (R)-10,11-dihydro-3-[3-(1-oxopyridin-2-ylamino)-1-propyloxy]-5H-dibenzo[a,d]cycloheptene-10-acetate

A solution of 2-[(3-hydroxy-1-propyl)amino]pyridine-N-oxide (0.70 g 4 mmole) and diethyl azodicarboxylate (0.65 mL, 4 mmole) in anhydrous DMF (20 mL) was added dropwise over 10 min to a solution of ethyl (R)-10,11-dihydro-3-hydroxy-5H-dibenzo[a,d]cycloheptene-10-acetate (0.45 g, 2 mmole) and triphenylphosphine (1.2 g, 4 mmole) in anhydrous DMF (8 mL) at RT under argon. After 23.5 hr, the reaction was concentrated on the rotavap, and the residue was reconcentrated from xylenes to remove residual DMF. Silica gel chromatography (1-4% CH₃OH/CH₂Cl₂) gave the title compound (0.50 g, 74%) as a yellow oil.

b) Ethyl (R)-10,11-dihydro-3-[3-(pyridin-2-ylamino)-1-propyloxy]-5H-dibenzo[a,d]cycloheptene-10-acetate

A mixture of ethyl (R)-10,11-dihydro-3-[3-(1-oxopyridin-2-ylamino)-1-propyloxy]-5H-dibenzo[a,d]cycloheptene-10-acetate (0.5 g, 1 mmol), 10% Pd/C (0.25 g, 0.2 mmole), cyclohexene (2 mL, 20 mmole), and isopropanol (10 mL) was heated at reflux for 18 hr, then the catalyst was removed by filtration through CELITE®. Silica gel chromatography (0.5-2% CH₃OH/CH₂Cl₂) gave the title compound (0.4 g, 83%) as a light yellow oil.

c) (R)-10,11-Dihydro-3-[3-(pyridin-2-ylamino)-1-propyloxy]-5H-dibenzo[a,d]cycloheptene-10-acetic acid

A mixture of ethyl (R)-10,11-dihydro-3-[3-(pyridin-2-ylamino)-1-propyloxyl-5H-dibenzo[a,d]cycloheptene-10-acetate (0.4 g, 0.93 mmole) and 1.0 N NaOH (1.1 mL. 1.1 mmole) in absolute EtOH (10 mL) was warmed in an oil bath set at 50° C. After 18 hr. the reaction was concentrated on the rotavap and the residue was dissolved in H₂O. The aqueous solution was adjusted to pH 4 with 3 N HCl, and the solid precipitate was collected and washed with H₂O. The material was dried in high vacuum at 40° C. to afford the title compound (0.36 g, 96%) as a nearly colorless solid.

### EXAMPLE 32

Preparation of (±)-10,11-dihydro-3-[3-(3,4,5,6-tetrahydropyrimidin-2-ylamino)-1-propyloxy]-5H-dibenzo[a,d]cycloheptene-10-acetic acid

a) Ethyl (±)-10,11-dihydro-3-[3-(4-nitrobenzyloxycarbonyl)amino-1-propyloxy]-5H-dibenzo[a,d]cycloheptene-10-acetate

According to the procedure of Example 31 (a), except substituting 3-(4-nitrobenzyloxycarbonylamino)-1-propanol for the 2-[(3-hydroxy-1-propyl)amino]pyridine-N-oxide, and ethyl (±)-10,11-dihydro-3-hydroxy-5H-dibenzo[a,d]cycloheptene-10-acetate for ethyl (R)-10,11-dihydro-3-hydroxy-5H-dibenzo[a,d]cycloheptene-10-acetate, the title compound was obtained as amber oil.

b) Ethyl (±)-10,11-dihydro-3-(3-amino-1-propyloxy)-5H-dibenzo[a,d]cycloheptene-10-acetate

A mixture of ethyl (±)-10,11-dihydro-3-[3-(4-nitrobenzyloxycarbonylamino)-1-propyloxy]-5H-dibenzo[a,d]cycloheptene-10-acetate (1.6 g, 3 mmol), 10% palladium on charcoal (0.8 g, 1 mmol), and ethanol (50 mL) was shaken under H₂ (48 psi) for 3 hr, then the catalyst was removed by filtration through CELITE®. The filtrate was concentrated to give the title compound (1.2 g, 100%) as a yellow oil.

c) Ethyl (±)-10,11-dihydro-3-[3-(pyrimidin-2-ylamino)-1-propyloxy]-5H-dibenzo[a,d]cycloheptene-10-acetate

A mixture of ethyl (±)-10,11-dihydro-3-(3-amino-1-propyloxy)-5H-dibenzo[a,d]cycloheptene-10-acetate (0.4 g, 1 mmole), sodium bicarbonate (0.5 g. 6 mmole), 2-bromopyrimidine (0.34 g, 2 mmole) and ethanol (10 mL) was heated at reflux under argon for 18 hr. The solution was then decanted and concentrated. The residue was purified by chromatography on silica gel (0.2-2% MeOH/CH₂Cl₂) to give the title compound (0.17 g, 34%) as a pale yellow oil.

d) Ethyl (±)-10,11-dihydro-3-[3-(3,4,5,6-tetrahydropyrimid-2-ylamino)-1-propyloxy]-5H-dibenzo[a,d]cycloheptene-10-acetate

A mixture of ethyl (±)-10,11-dihydro-3-[3-(pyrimidin-2-ylamino)-1-propyloxy]-5H-dibenzo[a,d]cycloheptene-10-acetate-(0.17g, 0.38 mmol), 10% palladium on charcoal (0.085g, 0.08 mmol), 4 M HCl in dioxane (0.1 mL, 0.4 mmol) and ethanol (5 mL) was shaken under H₂ (48 psi) for 6 hr, then the catalyst was removed by filtration through CELITE®. The filtrate was concentrated to give the title compound (0.19 g) as a yellow oil.

e) (±)-10,11-Dihydro-3-[3-(3,4,5,6-tetrahydropyrimid-2-ylamino)-1-propyloxy]-5H-dibenzo[a,d]cycloheptene-10-acetic acid

A solution of ethyl (±)-10,11-dihydro-3-[3-(3,4,5,6-tetrahydropyrimid-2-ylamino)-1-propyloxy]-5H-dibenzo[a,d]cycloheptene-10-acetate (0.17g, 0.36 mmol), lithium hydroxide monohydrate (0.042 g, 1 mmol), THF (3 mL), and water (10 mL) was stirred at room temperature for 20 hr, then was concentrated. The residue was dissolved in water, and the solution was brought to pH 4 with 3 N HCl. The resulting solution was kept in the refrigerator overnight, then the supernatant was decanted away from the solid. The solid was dried in vacuum to give the title compound (0.145 g, 91%) as a tan solid.

### EXAMPLE 33

Parenteral Dosage Unit Composition

A preparation which contains 50 mg of the compound of Example 1 as a sterile dad powder is prepared as follows: 20 mg of the compound is dissolved in 15 mL of distilled water. The solution is filtered under sterile conditions into a 25 mL multi-dose ampoule and lyophilized. The powder is reconstituted by addition of 20 mL of 5% dextrose in water (D5W) for intravenous or intramuscular injection. The dosage is thereby determined by the injection volume. Subsequent dilution may be made by addition of a metered volume of this dosage unit to another volume of D5W for injection, or a metered dose may be added to another mechanism for dispensing the drug, as in a bottle or bag for IV drip infusion or other injection-infusion system.

### EXAMPLE 34

Oral Dosage Unit Composition

A capsule for oral administration is prepared by mixing and milling 50 mg of the compound with 75 mg of lactose and 5 mg of magnesium stearate. The resulting powder is screened and filled into a hard gelatin capsule.

### EXAMPLE 35

Oral Dosage Unit Composition

A tablet for oral administration is prepared by mixing and granulating 20 mg of sucrose, 150 mg of calcium sulfate dihydrate and 100 mg of the compound with a 10% gelatin solution. The wet granules are screened, dried, mixed with 10 mg starch, 5 mg talc and 3 mg stearic acid; and compressed into a tablet.

The above description fully discloses how to make and use this invention. This invention, however, is not limited to the precise embodiments described herein, but encompasses all modifications within the scope of the claims which follow.

What is claimed is:

1. A method for stimulating bone formation in a mammal which comprises administering a compound which stimulates osteocalcin release.

2. A method according to claim 1 wherein the compound stimulates osteocalcin release with an EC50 of less than 5 uM in the ROS 17/2.8 osteocalcin assay.

3. A method according to claim 1 wherein the compound binds to an integrin receptor.

4. A method according to claim 3 wherein the compound binds to the αᵥβ₃ receptor.

5. A method according to claim 4 wherein the compound binds to the vitronectin receptor with a Ki of less than 1 uM.

6. A method according to claim 3 wherein the compound is of formula (I) or (II): 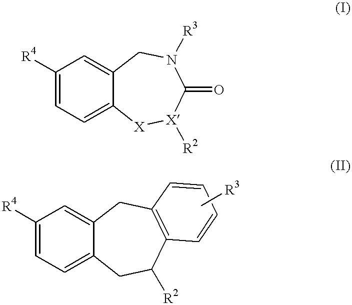
wherein
X-X′ is NR¹-CH, NC(O)R¹-CH, N═C, CR¹═C, CHR¹-CH, O—CH or S—CH;
R¹ is H, C₁₋₆ alkyl or Ar-C₁₋₆alkyl;
R² is (CH₂)ₙCO₂R′;
R³ is H, C₁₋₆alkyl, Ar-C₀₋₆alkyl, Het-C₀₋₆alkyl, or C₃₋₆cycloalkyl-C₀₋₆alkyl;
R⁴ is W-(Q′)ₚ—(CR′₂)_{q}—U—(CR′₂)ₛ-;
R⁵ and R⁶ are H, C₁₋₆alkyl, Ar-C₀₋₆alkyl, Het-C₀₋₆alkyl or C₃₋₆cycloalkyl-C₀₋₆alkyl;
R′ is H, C₁₋₆alkyl, C₃₋₇cycloalkyl-C₀₋₄alkyl or Ar-C₀₋₄alkyl;
Q′ is NR⁵, S or CR⁵;
U is NR⁶C(O), C(O)NR⁶, CH₂CO, COCH₂, CH═CH, C≡C, CH₂—CH₂, O—CH₂, CH₂—O, O, S, NR⁶, C(O), CH₂ or CH₂OCONR¹; 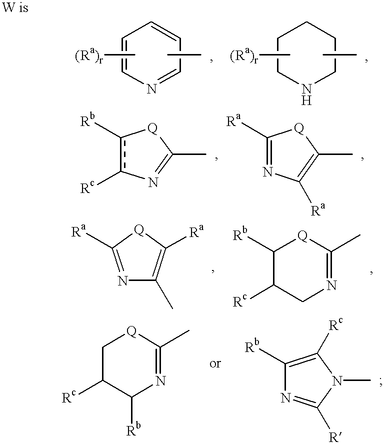
Q is NR′, O or S; [symbol]
indicates a single or double bond:
R^{a} is independently H, C₁₋₆alkyl, Ar—C₀₋₆alkyl, Het—C₀₋₆alkyl, or C₃₋₆cycloalkyl—C₀₋₆alkyl, halogen, OR¹, SR¹, COR¹, OH, NO₂, N(R¹)₂, CO(NR¹)₂, CH₂N(R¹)₂ or R¹HN—C(=NH);
R^{b} and R^{c} are independently selected from H, C₁₋₆alkyl, Ar-C₀₋₆alkyl, Het-C₀₋₆alkyl, or C₃₋₆cycloalkyl-C₀₋₆alkyl, halogen, OR¹, SR¹, COR¹, OH, NO₂, N(R¹)₂, CO(NR¹)₂, CH₂N(R¹)₂, or R^{b} and R^{c} are joined together to form a five or six membered aromatic or non-aromatic ring, optionally substituted by halogen, C₁₋₄alkyl, OR¹, SR¹, COR¹, OH, NO₂, N(R¹)₂, CO(NR¹)₂, CH₂N(R¹)₂ or R¹HN—C(=NH); or
n is 1 or 2;
p is 0 or 1;
q is 0, 1, 2 or 3;
r is 0, 1 or 2;
s is 0, 1, 2 or 3; or a pharmaceutically acceptable salt thereof.

7. A method according to claim 3 wherein the compound is selected from the group of:
(+/−)-7-[[(2-Methyl benzimidazoloyl)amino]carbonyl]-2,3,4,5-tetrahydro-4-methyl-3-oxo-1H-1,4-benzodiazepine-2-acetic acid;
(+/−)-7-[[(2-Methyl benzimidazoloyl)amino]carbonyl]-2,3,4,5-tetrahydro-4-methyl-3-oxo-1H-1,4-benzodiazepine-2-acetic acid;
(S)-(-)-7-[[[(2-Benzimidazolyl)methyl]amino]carbonyl]-2,3,4,5-tetrahydro-4-methyl-3-oxo-1H-1,4-benzodiazepine-2-acetic acid;
(+/−)-7-[[[(2-Methylbenzimidazolyl](N-methyl)amino]carbonyl]-2,3,4,5-tetrahydro-4-methyl-3-oxo-1H-1,4-benzodiazepine-2-acetic acid trifluoroacetate salt;
(+/−)-7-[[[(Benzimidazol-2-yl)methyl]amino]carbonyl]-3-oxo-4-(2-phenylethyl)-2,3,4,5-tetrahydro-1H-1,4-benzodiazepine-2-acetic acid;
(+/−)-7-[[[(Benzimidazol-2-yl)methyl]amino]carbonyl]-3-oxo-4-isopropyl-2,3,4,5-tetrahydro-1H-1,4-benzodiazepine-2-acetic acid trifluoroacetate salt;
(+/−)-2-Methyl-3-oxo-8-( [(2-benzimidazoyl)methylamino]carbonyl)-2,3,4,5-tetrahydro-2-benzazepine-4-acetic acid;
(+/−)-8-[[(2-Benzimidazolylmethyl](N-methyl)amino]carbonyl]-2,3,4,5-tetrahydro-2-methyl-3-oxo-2-benzapine-4-acetic acid hydrochloride salt;
(+/−)-7-[[[(2-Benzimidazolylmethyl](N-methyl)amino]carbonyl]-2,3,4,5-tetrahydro-3-oxo-4-(2-phenylethyl)-1H-1,4-benzodiazepine-2-acetic acid;
(S)-7-[[(1H-Benzimidazol-2-ylmethyl)methylamino]carbonyl]-2,3,4,5-tetrahydro-4-methyl-3-oxo-1H-1,4-benzodiazepine-2-acetic acid=(S)-(-)-7-[[N-alpha-[(2-Methylbenzimidazolyl) (N-methyl)amino]carbonyl]-2,3,4,5-tetrahydro-4-methyl-3-oxo-1H-1,4-benzodiazepine-2-acetic acid;
(+/−)-7-[[[(1-N-methyl)-2-methylbenzimidazolyl](N-methyl)amino]carbonyl]-2,3,4,5-tetrahydro-4-methyl-3-oxo-1H-1,4-benzodiazepine-2-acetic acid;
(S)-7-[[(2-Benzimidazol-2-ylmethyl](N-methyl)amino]carbonyl]-2,3,4,5-tetrahydro-3-oxo-4-(2-phenylethyl)-1H-1,4-benzodiazepine-2-acetic acid;
(-)-7-[[[5,6-Methylenedioxybenzimidazol-2-ylmethyl]aminomethyl]carbonyl]-2,3,4,5-tetrahydro-4-methyl-3-oxo-1,4-benzodiazepine-2-acetic acid;
(2S)-7-[[[N-Butyl-N-(benzimidazol-2-yl)methyl]amino]carbonyl]-3-oxo-4-methyl-2,3,4,5-tetrahydro-1H-1,4-benzodiazepine-2-acetic acid;
(−)-7-[[[Imidazo[4,5B]pyridyl-2-ylmethyl]aminomethyl carbonyl]2,3,4,5-tetrahydro-4-methyl-3-oxo-1,4-benzodiazepine-2-acetic acid;
(2S)-[[[N-Phenylethyl-N-(benzimidazol-2-yl)methyl]amino]carbonyl]-3-oxo-4-methyl-2,3,4,5-tetrahydro-1H-1,4-benzodiazepine-2-acetic acid;
(+/−)-7-[[[(2-Methyl)benzimidazolyl](N-methyl)amino]carbonyl]-2,3,4,5-tetrahydro-3-oxo-1H-1,4-benzodiazepine-2-acetic acid trifluoroacetate;
(+/−)-7-[[[N-Benzimidazol-2-yl]methyl]amino]methyl]-4-methyl-3-oxo-2,3,4,5-tetrahydro-1H-1,4-benzodiazepine-2-acetic acid bis(trifluoroacetate);
(+/−)-7-[[[Imidazo[4,5-B ]pyridyl-2-ylmethyl]aminomethyl]carbonyl]2,3,4,5-tetrahydro-4-isoproyl-3-oxo-1,4-benzodiazepine-2-acetic acid;
(−)-7-[[[Imidazo[4,5-B]-6-methylpyridyl-2-ylmethyl]aminomethyl]carbonyl]-2,3,4,5-tetrahydro-4-methyl-3-oxo-benzodiazepine-2-acetic acid.;
(+/−)-2,3,4,5-Tetrahydro-7-[[[(benzimidazol-2-yl)methyl]methylamino]carbonyl]-4-(2-methoxyethyl)-3-oxo-1H-1,4-benzodiazepine-2-acetic acid;
(+/−)-2,3,4,5-Tetrahydro-7-[[[[imidazo[4,5-B]pyridyl-2-yl]methyl]methylamino]carbonyl]-4-(2-methoxyethyl)-3-oxo-1H-1,4-benzodiazepine-2-acetic acid trifluoroacetate salt;
(+/−)-7-[[(2-Benzimidazol-2-ylmethyl)amino]carbonyl]-2,3,4,5-tetrahydro-3-oxo-4-[2-(3′,4′-methylenedioxyphenyl)ethyl]-1H-1,4-benzodiazepine-2-acetic acid; and
(±)-2,3,4,5-Tetrahydro-3-oxo-4-(phenylethyl)-7-[[[2-[2-(pyridinyl)amino]ethyl]amino]carbonyl]-1H-1,4-benzodiazepine-2-acetic acid.

8. A method according to claim 3 wherein the compound is selected from the compounds of formula (III)-(X) as described herein.

9. A method according to claim 3 wherein the binding to the α_{IIb}β₃ receptor is greater than 10 uM.

10. A method according to claim 1 for treating or preventing bone fractures.

11. A method according to claim 1 for treating osteoporosis, hyperparathyroidism, Paget's disease, hypercalcemia of malignancy, osteolytic lesions produced by bone metastasis, or bone loss due to immobilization or sex hormone deficiency.

12. An assay for detecting compounds which stimulate mineralization or bone formation which comprises treating an osteoblastic cell with a compound and assaying for osteocalcin release.

13. An assay according to claim 12 wherein the osteoblastic cell is a ROS 17/2.8 cell.

14. An assay according to claim 12 wherein the compound is incubated with the osteoblasts for at least 24 hours.

15. An assay according to claim 12 wherein the osteocalcin is quantitated with a radioimmunoassay.
